(19) United States
(12) Patent Application Publication
(10) Pub. No.: US 2024/0018251 A1
(43) Pub. Date: Jan. 18, 2024

(54) BCMA-TARGETING SINGLE-DOMAIN ANTIBODY AND USE THEREOF

(71) Applicant: BIOHENG THERAPEUTICS LIMITED, GRAND CAYMAN (KY)

(72) Inventors: Guokun LI, NANJING (CN); Rongrong PU, NANJING (CN); Jiangtao REN, NANJING (CN); Xiaohong He, Nanjing (CN); Yanbin Wang, Nanjing (CN); Lu HAN, NANJING (CN)

(73) Assignee: BIOHENG THERAPEUTICS LIMITED, GRAND CAYMAN (KY)

(21) Appl. No.: 18/034,167

(22) PCT Filed: Oct. 25, 2021
(86) PCT No.: PCT/CN2021/126043

§ 371 Date: Apr. 27, 2023

(30) Foreign Application Priority Data
Oct. 30, 2020 (CN) 202011194599.9

(51) Int. Cl. C07K 16/28; C12N 15/63; A61P 35/00

(52) U.S. Cl. CPC C07K 16/2878; C12N 15/63; A61P 35/00; C07K 2317/565; C07K 2317/567; C07K 2317/569; C07K 2317/24; C07K 2317/31

(57) ABSTRACT

Provided in the present invention are a BCMA-targeting single-domain antibody and an encoding nucleotide sequence thereof. Also provided in the present invention are a multispecific antibody, a chimeric antigen receptor, and an antibody conjugate containing the BCMA single domain antibody, and a pharmaceutical composition and kit containing said antibodies, chimeric antigen receptor and antibody conjugate, and the use thereof in the diagnosis/treatment/prevention of diseases associated with BCMA expression.

### TECHNICAL FIELD

The present disclosure belongs to the field of immunotherapy. More specifically, the present disclosure relates to a BCMA-targeting single domain antibody and use thereof in the prevention and/or treatment and/or diagnosis of diseases.

### BACKGROUND ART

BCMA (B cell maturation antigen), also known as CD269 or TNFRSF17, is a member of the tumor necrosis factor receptor (TNFR) superfamily, and can specifically bind to B cell-activating factor (BAFF) or proliferation-inducing ligand (APRIL). BCMA is mainly expressed in plasma cells and mature B cells. It has been reported that the expression of BCMA is associated with several diseases, such as cancer, autoimmune diseases, infectious diseases and so on. BCMA is a potential therapeutic target due to its important role in various diseases and conditions, for example, cancer.

The present disclosure aims to provide an anti-BCMA single domain antibody and an antibody conjugate, a chimeric antigen receptor, a multispecific antibody, a pharmaceutical composition containing the anti-BCMA single domain antibody, and use thereof in the prevention and/or treatment and/or diagnosis of diseases.

### SUMMARY

In an aspect, the present disclosure provides an anti-BCMA single domain antibody comprising three complementarity determining regions CDR1, CDR2 and CDR3, wherein CDR1 is selected from the group consisting of SEQ ID NO: 1, SEQ ID NO: 4 and SEQ ID NO: 7, CDR2 is selected from the group consisting of SEQ ID NO: 2 and SEQ ID NO: 5, and CDR3 is selected from the group consisting of SEQ ID NO: 3 and SEQ ID NO: 6.

In an embodiment, the anti-BCMA single domain antibody comprises:
(1) CDR1 as set forth in SEQ ID NO: 1, CDR2 as set forth in SEQ ID NO: 2, and CDR3 as set forth in SEQ ID NO: 3; or
(2) CDR1 as set forth in SEQ ID NO: 4 or SEQ ID NO: 7, CDR2 as set forth in SEQ ID NO: 5, and CDR3 as set forth in SEQ ID NO: 6.

In an embodiment, the anti-BCMA single domain antibody of the present disclosure comprises four framework regions FR1, FR2, FR3 and FR4, wherein FR1 is selected from the group consisting of SEQ ID NO: 8, SEQ ID NO: 12, SEQ ID NO: 17, SEQ ID NO: 20, SEQ ID NO: 22 or variants thereof, FR2 is selected from the group consisting of SEQ ID NO: 9, SEQ ID NO: 13, SEQ ID NO: 18 or variants thereof, FR3 is selected from the group consisting of SEQ ID NO: 10, SEQ ID NO: 14, SEQ ID NO: 19, SEQ ID NO: 21, SEQ ID NO: 23 or a variants thereof, and FR4 is selected from the group consisting of SEQ ID NO: 11, SEQ ID NO: 15, SEQ ID NO: 16, SEQ ID NO: 24 or variants thereof, wherein the variant comprises substitution of at most 3 amino acids, preferably conservative substitution of at most 3 amino acids in the FR.

In an embodiment, the anti-BCMA single domain antibody comprises:
(1) 1-R1 as set forth in SEQ ID NO: 8, FR2 as set forth in SEQ ID NO: 9, FR3 as set forth in SEQ ID NO: 10, FR4 as set forth in SEQ ID NO: 11, or variants thereof, wherein the variant comprises substitution of at most 3 amino acids in the FR;
(2) FR1 as set forth in SEQ ID NO: 12 or SEQ ID NO: 22, FR2 as set forth in SEQ ID NO: 13, FR3 as set forth in SEQ ID NO: 14, FR4 as set forth in SEQ ID NO: 15 or SEQ ID NO: 16, or variants thereof, wherein the variant comprises substitution of at most 3 amino acids in the FR;
(3) FR1 as set forth in SEQ ID NO: 17, FR2 as set forth in SEQ ID NO: 18, FR3 as set forth in SEQ ID NO: 19, FR4 as set forth in SEQ ID NO: 15, or variants thereof, wherein the variant comprises substitution of at most 3 amino acids in the FR;
(4) FR1 as set forth in SEQ ID NO: 20, FR2 as set forth in SEQ ID NO: 13, FR3 as set forth in SEQ ID NO: 21, FR4 as set forth in SEQ ID NO: 15 or SEQ ID NO: 16, or variants thereof, wherein the variant comprises substitution of at most 3 amino acids in the FR; or
(5) 1-R1 as set forth in SEQ ID NO: 8, FR2 as set forth in SEQ ID NO: 9, FR3 as set forth in SEQ ID NO: 23, FR4 as set forth in SEQ ID NO: 11 or SEQ ID NO: 24, or variants thereof, wherein the variant comprises substitution of at most 3 amino acids in the FR.

In an embodiment, the anti-BCMA single domain antibody has at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NO: 25-33, and specifically bind to BCMA antigen. Preferably, the amino acid sequence of the anti-BCMA single domain antibody is set forth in SEQ ID NO: 25-33.

In an embodiment, the anti-BCMA single domain antibody is a natural Camelidae antibody or a chimeric antibody, such as a camelized antibody, a humanized antibody or a human antibody, preferably a humanized antibody. Preferably, the humanized anti-BCMA single domain antibody comprises FR1 selected from the group consisting of SEQ ID NO: 43, SEQ ID NO: 46, SEQ ID NO: 50, SEQ ID NO: 52, SEQ ID NO: 55, SEQ ID NO: 56, SEQ ID NO: 57, SEQ ID NO: 60, SEQ ID NO: 67 or variants thereof, FR2 selected from the group consisting of SEQ ID NO: 9, SEQ ID NO: 13, SEQ ID NO: 47, SEQ ID NO: 61 or variants thereof, FR3 selected from the group consisting of SEQ ID NO: 44, SEQ ID NO: 48, SEQ ID NO: 51, SEQ ID NO: 53, SEQ ID NO: 54, SEQ ID NO: 58, SEQ ID NO: 62, SEQ ID NO: 65, SEQ ID NO: 66, SEQ ID NO: 68, SEQ ID NO: 69 or variants thereof, and FR4 selected from the group consisting of SEQ ID NO: 15, SEQ ID NO: 24, SEQ ID NO: 45, SEQ ID NO: 49, SEQ ID NO: 59, SEQ ID NO: 63, SEQ ID NO: 64, SEQ ID NO: 70, SEQ ID NO: 71 or variants thereof, wherein the variant comprises substitution of at most 3 amino acids in the FR.

In an embodiment, the humanized anti-BCMA single domain antibody comprises:
(1) FR1 as set forth in SEQ ID NO: 43, SEQ ID NO: 52, SEQ ID NO: 55 or SEQ ID NO: 56, FR2 as set forth in SEQ ID NO: 13, FR3 as set forth in SEQ ID NO: 44, SEQ ID NO: 51, SEQ ID NO: 53 or SEQ ID NO: 54, FR4 as set forth in SEQ ID NO: 15 or SEQ ID NO: 45, or variants thereof, wherein the variant comprises substitution of at most 3 amino acids in the FR;
(2) FR1 as set forth in SEQ ID NO: 46 or SEQ ID NO: 50, FR2 as set forth in SEQ ID NO: 47, FR3 as set forth in SEQ ID NO: 48 or SEQ ID NO: 51, FR4 as set forth in SEQ ID NO: 49, or variants thereof, wherein the variant comprises substitution of at most 3 amino acids in the FR;
(3) FR1 as set forth in SEQ ID NO: 57 or SEQ ID NO: 67, FR2 as set forth in SEQ ID NO: 9, FR3 as set forth in SEQ ID NO: 58, SEQ ID NO: 65, SEQ ID NO: 66, SEQ ID NO: 68 or SEQ ID NO: 69, FR4 as set forth in SEQ ID NO: 24, SEQ ID NO: 59, SEQ ID NO: 63, SEQ ID NO: 64, SEQ ID NO: 70 or SEQ ID NO: 71, or variants thereof, wherein the variant comprises substitution of at most 3 amino acids in the FR; or
(4) FR1 as set forth in SEQ ID NO: 60, FR2 as set forth in SEQ ID NO: 61, FR3 as set forth in SEQ ID NO: 62, FR4 as set forth in SEQ ID NO: 63, or variants thereof, wherein the variant comprises substitution of at most 3 amino acids in the FR.

More preferably, the humanized anti-BCMA single domain antibody has at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NO: 72-86. Preferably, the amino acid sequence of the anti-BCMA single domain antibody is set forth in SEQ ID NO: 72-86.

The present disclosure further provides a nucleic acid molecule encoding the anti-BCMA single domain antibody. Therefore, in an embodiment, the nucleic acid molecule encoding the anti-BCMA single domain antibody has at least 90%, at least 91%, at least 92%, at least 93%, at least 94%, at least 95%, at least 96%, at least 97%, at least 98%, at least 99%, or 100% sequence identity to a nucleotide sequence selected from the group consisting of SEQ ID NO: 34-42 and 87-101, and the anti-BCMA single domain antibody encoded can specifically bind BCMA antigen. Preferably, the nucleic acid molecule encoding the anti-BCMA single domain antibody is set forth in SEQ ID NO: 34-42 and 87-101.

In another aspect, the present disclosure further provides a multispecific antibody (preferably bispecific antibody or trispecific antibody), which comprises the anti-BCMA single domain antibody (including humanized single domain antibody) as described above, and one or more second antibodies or antigen-binding portions thereof that specifically bind to antigens different from BCMA.

In an embodiment, the second antibody or antigen-binding portion thereof may be in the form of any antibody or antibody fragment, such as a full-length antibody, Fab, Fab′, (Fab′)₂, Fv, scFv, scFv-scFv, a minibody, a diabody or sdAb.

The present disclosure further provides a vector comprising a nucleic acid molecule encoding the anti-BCMA single domain antibody or the multispecific antibody as described above, and a host cell expressing the anti-BCMA single domain antibody or the multispecific antibody.

In another aspect, the present disclosure further provides a chimeric antigen receptor, which comprises the anti-BCMA single domain antibody of the present disclosure, a transmembrane domain and an intracellular signaling domain. Preferably, the chimeric antigen receptor further comprises one or more co-stimulatory domains. More preferably, the chimeric antigen receptor comprises the anti-BCMA single domain antibody (including humanized single domain antibody) or the multispecific antibody comprising the anti-BCMA single domain antibody as provided herein, a CD8a transmembrane region, a CD28 or 4-1BB co-stimulatory domain, and a CD3ζ intracellular signaling domain.

The present disclosure further provides a nucleic acid molecule encoding the BCMA-targeting chimeric antigen receptor as defined above, and a vector comprising the nucleic acid molecule.

The present disclosure further provides a cell, preferably an immune cell, such as a T cell, a NK cell, a NKT cell, a macrophage, and a dendritic cell, comprising the BCMA-targeting chimeric antigen receptor as defined above.

In another aspect, the present disclosure further provides an antibody conjugate comprising the anti-BCMA single domain antibody defined in the present disclosure and a second functional structure, wherein the second functional structure is selected from the group consisting of Fc, a radioisotope, a structure moiety for extending half-life, a detectable marker and a drug.

In an embodiment, the structure moiety for extending half-life is selected from the group consisting of an albumin-binding structure, a transferrin-binding structure, a polyethylene glycol molecule, a recombinant polyethylene glycol molecule, human serum albumin, a fragment of human serum albumin, and a polypeptide (including an antibody) binding to human serum albumin. In an embodiment, the detectable marker is selected from the group consisting of a fluorophore, a chemiluminescent compound, a bioluminescent compound, an enzyme, an antibiotic resistance gene, and a contrast agent. In an embodiment, and the drug is selected from the group consisting of a cytotoxin and an immunomodulator. In another aspect, the present disclosure further provides a detection kit comprising the single domain antibody, the multispecific antibody, the antibody conjugate or the chimeric antigen receptor described in the present disclosure.

In another aspect, the present disclosure further provides a pharmaceutical composition comprising the single domain antibody, the chimeric antigen receptor, the multispecific antibody or the antibody conjugate described in the present disclosure, and one or more pharmaceutically acceptable excipients.

In another aspect, the present disclosure further provides a method for treating and/or preventing and/or diagnosing diseases associated with BCMA expression, comprising administering to a subject the single domain antibody, the chimeric antigen receptor, the multispecific antibody, the antibody conjugate or the pharmaceutical composition as described above. Preferably, the disease associated with BCMA expression is selected from the group consisting of autoimmune diseases, lymphoma, leukemia or plasma cell malignancies.

### DETAILED DESCRIPTION OF EMBODIMENTS

Unless otherwise specified, all scientific and technical terms used herein have the same meaning as commonly understood by one of ordinary skill in the art to which this disclosure belongs.

Anti-BCMA Single Domain Antibody

As used herein, the term “single domain antibody” or “sdAb” has the same meaning and refers to a single immunoglobulin variable domain (V_{H}, V_{HH}, or V_{L}) polypeptide having three complementarity determining regions (CDRs) that specifically binds an antigen. They are capable of binding target antigen without the presence of the corresponding CDR-containing light chain/heavy chain partners or other parts of intact antibodies. Single-domain antibodies derived from camelid heavy-chain-only antibodies that naturally lack light chains, as well as single-domain antibodies with human heavy-chain domains (Muyldermans 2001, Holliger 2005), and single V_{H} domains identified from a murine V_{H} gene library amplified from genomic DNA from the spleen of immunized mice (Ward et al., 1989, Nature 341:544-546) have been reported. Single-domain antibodies usually have the following structure from N-terminus to C-terminus: FR1-CDR1-FR2-CDR2-FR3-CDR3-FR4, where FR1 to FR4 refer to framework regions 1 to 4, respectively, and CDR1 to CDR3 refer to complementarity determining regions 1 to 3.

The term “complementarity determining region” or “CDR” is well known to those skilled in the art and used interchangeably, and refers to the non-contiguous sequence of amino acids within the variable region of an antibody that confers antigen specificity and/or binding affinity. The term “framework region” or “FR” is also known in the art and refers to the non-CDR portion of the antibody variable region, the sequence of which is generally conserved.

The precise amino acid sequence boundaries for a given CDR or FR can be readily determined using a number of numbering schemes well known in the art, including: Kabat et al. (1991), “Sequences of Proteins of Immunological Interest,” 5th Edition, Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol 262:732-745 (1996), “Antibody-antigen interactions: Contact analysis and binding sitetopography,” J. Mol. Biol. 262, 732-745″ (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pltickthun A, “Yet another numbering scheme for immunoglobulin variable domains: an automatic modeling and analysis tool,” JMol Biol, 2001 Jun. 8; 309(3):657-70 (“Aho” numbering scheme); and Martin et al., “Modeling antibody hypervariable loops: a combined algorithm,” PNAS, 1989, 86(23):9268-9272 (“AbM” numbering scheme).

The boundaries of a given CDR or FR may vary depending on the scheme used for identification. For example, the Kabat scheme is based on structural alignments, while the Chothia scheme is based on structural information. Both the Kabat and Chothia numbering schemes are based on the sequence lengths of the most common antibody regions where insertions are provided by caret letters (e.g., “30a”) and deletions occur in some antibodies. These two schemes place certain insertions and deletions (“indels”) at different positions, resulting in different numbering. The Contact scheme is based on the analysis of complex crystal structures and is similar in many respects to the Chothia numbering scheme. The AbM scheme is a compromise between the Kabat and Chothia definitions and is based on the scheme used by the AbM antibody modeling software of Oxford Molecular.

Thus, unless otherwise specified, a “CDR” of a given antibody or region thereof (e.g., variable region thereof) is understood to encompass the CDRs defined by any of the above schemes or other known schemes. For example, where it is specified that a particular CDR (e.g., CDR3) contains a given amino acid sequence, it is understood that such a CDR may also have the sequence of the corresponding CDR (e.g., CDR3) as defined by any of the above schemes or other known schemes. Likewise, unless otherwise specified, FRs for a given antibody or region thereof (e.g., variable region thereof) are understood to encompass 1-Rs as defined by any of the above schemes or other known schemes.

Therefore, in an aspect, the present disclosure provides an anti-BCMA single domain antibody comprising three complementarity determining regions CDR1, CDR2 and CDR3, wherein CDR1 is selected from the group consisting of SEQ ID NO: 1, SEQ ID NO: 4 and SEQ ID NO: 7, CDR2 is selected from the group consisting of SEQ ID NO: 2 and SEQ ID NO: 5, and CDR3 is selected from the group consisting of SEQ ID NO: 3 and SEQ ID NO: 6.

In an embodiment, the anti-BCMA single domain antibody comprises:
(1) CDR1 as set forth in SEQ ID NO: 1, CDR2 as set forth in SEQ ID NO: 2, and CDR3 as set forth in SEQ ID NO: 3; or
(2) CDR1 as set forth in SEQ ID NO: 4 or SEQ ID NO: 7, CDR2 as set forth in SEQ ID NO: 5, and CDR3 as set forth in SEQ ID NO: 6.

In an embodiment, the anti-BCMA single domain antibody of the present disclosure comprises four framework regions FR1, FR2, FR3 and FR4, wherein FR1 is selected from the group consisting of SEQ ID NO: 8, SEQ ID NO: 12, SEQ ID NO: 17, SEQ ID NO: 20, SEQ ID NO: 22 or variants thereof, FR2 is selected from the group consisting of SEQ ID NO: 9, SEQ ID NO: 13, SEQ ID NO: 18 or variants thereof, FR3 is selected from the group consisting of SEQ ID NO: 10, SEQ ID NO: 14, SEQ ID NO: 19, SEQ ID NO: 21, SEQ ID NO: 23 or variants thereof, and FR4 is selected from the group consisting of SEQ ID NO: 11, SEQ ID NO: 15, SEQ ID NO: 16, SEQ ID NO: 24 or variants thereof, wherein the variant comprises substitution of at most 3 amino acids, preferably conservative substitution of at most 3 amino acids in the FR.

In an embodiment, the anti-BCMA single domain antibody comprises:
(1) 1-R1 as set forth in SEQ ID NO: 8, FR2 as set forth in SEQ ID NO: 9, FR3 as set forth in SEQ ID NO: 10, FR4 as set forth in SEQ ID NO: 11, or variants thereof, wherein the variant comprises substitution of at most 3 amino acids in the FR;
(2) FR1 as set forth in SEQ ID NO: 12 or SEQ ID NO: 22, FR2 as set forth in SEQ ID NO: 13, FR3 as set forth in SEQ ID NO: 14, FR4 as set forth in SEQ ID NO: 15 or SEQ ID NO: 16, or variants thereof, wherein the variant comprises substitution of at most 3 amino acids in the FR;
(3) FR1 as set forth in SEQ ID NO: 17, FR2 as set forth in SEQ ID NO: 18, FR3 as set forth in SEQ ID NO: 19, FR4 as set forth in SEQ ID NO: 15, or variants thereof, wherein the variant comprises substitution of at most 3 amino acids in the FR;
(4) FR1 as set forth in SEQ ID NO: 20, FR2 as set forth in SEQ ID NO: 13, FR3 as set forth in SEQ ID NO: 21, FR4 as set forth in SEQ ID NO: 15 or SEQ ID NO: 16, or variants thereof, wherein the variant comprises substitution of at most 3 amino acids in the FR; or
(5) 1-R1 as set forth in SEQ ID NO: 8, FR2 as set forth in SEQ ID NO: 9, FR3 as set forth in SEQ ID NO: 23, FR4 as set forth in SEQ ID NO: 11 or SEQ ID NO: 24, or variants thereof, wherein the variant comprises substitution of at most 3 amino acids in the FR.

In an embodiment, the anti-BCMA single domain antibody has at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NO: 25-33, and specifically binds to BCMA antigen. Preferably, the amino acid sequence of the anti-BCMA single domain antibody is set forth in SEQ ID NO: 25-33.

As used herein, the term “conservative substitution” refers to an amino acid substitution that does not significantly affect or alter the binding characteristics of an antibody or antibody fragment comprising the amino acid sequence Amino acid substitutions can be introduced into the antibodies of the present disclosure by standard techniques known in the art, such as site-directed mutagenesis and PCR-mediated mutagenesis. A conservative amino acid substitution is one in which an amino acid residue is replaced by an amino acid residue with a similar side chain Families of amino acid residues with similar side chains have been defined in the art and include those with basic side chain (e.g., lysine, arginine, histidine), acidic side chain (e.g., aspartic acid, glutamic acid), uncharged polar side chain (e.g., glycine, asparagine, glutamine, serine, threonine, tyrosine, cysteine), non-polar side chain (e.g., alanine, valine, leucine, isoleucine, proline, phenylalanine, methionine, tryptophan), beta-branched side chain (e.g., threonine, valine, isoleucine) and aromatic side chain (e.g., tyrosine, phenylalanine, tryptophan, histidine). Conservative modifications can be selected, for example, on the basis of similarity in polarity, charge, solubility, hydrophobicity, hydrophilicity, and/or the amphipathic nature of the residues involved.

As used herein, the term “sequence identity” means the degree to which two (nucleotide or amino acid) sequences in alignment have the same residue at the same position, and is usually expressed as a percentage. Preferably, identity is determined over the entire length of the sequences being compared. Therefore, two copies of the exact same sequence have 100% identity. Those skilled in the art know that several algorithms can be used to determine sequence identity, such as Blast (Altschul et al. (1997) Nucleic Acids Res. 25:3389-3402), Blast2 (Altschul et al. (1990) J. Mol. Biol. 215: 403-410), Smith-Waterman (Smith et al. (1981) J. Mol. Biol. 147:195-197) and Clustal W.

As used herein, the term “variant” or “functional fragment” has at most 10 (for example, 1, 2, 3, 4, 5, 6, 7, 8, 9, or 10) amino acid substitutions compared with the parental amino acid sequence, or has 90%, 91%, 92%, 93%, 94%, 95%, 96%, 97%, 98%, or 99% sequence identity to the parental amino acid sequence, and retain the biological activity of the parent amino acid sequence, such as binding activity.

Examples of single domain antibodies include, but are not limited to, heavy chain variable domains from heavy chain antibodies, binding molecules naturally devoid of light chains, single domains derived from conventional four chain antibodies (e.g., V_{H} or V_{L}), humanized heavy chain antibodies, human single domain antibodies produced by transgenic mice or rats expressing human heavy chain fragments, among others. Single domain antibodies can be from any species including, but not limited to, mouse, rat, human, camel, llama, lamprey, fish, shark, goat, rabbit, and cow.

In an embodiment, the single domain antibody is a single domain antigen binding molecule derived from a naturally occurring heavy chain antibody (also known as HCAb). For example, single domain antibodies can be derived from species of the family Camelidae, such as camel, llama, vicuna, dromedary, alpaca, and vicugna. Single-domain antibodies derived from Camelidae, also known as V_{HH}, have a molecular weight of about 15 kD and are considered to be the smallest functional antigen-binding fragment.

In some embodiments, single domain antibodies are derived from the variable regions of immunoglobulins found in cartilaginous fish. For example, single domain antibodies can be derived from an immunoglobulin isotype called neoantigen receptor (NAR) found in shark serum.

In some embodiments, the single domain antibody is a human single domain antibody produced by a transgenic mouse or rat expressing a human heavy chain fragment. See e.g. US20090307787A1, U.S. Pat. No. 8,754,287, US20150289489A1, US20100122358A1 and WO 2004049794.

In some embodiments, single domain antibodies may also be obtained from (natural or immune) libraries of Camelidae V_{HH} sequences. Such methods include, for example, screening such libraries using the corresponding antigen or fragment, antigenic determinant or epitope, etc., by screening techniques known in the art. Alternatively, improved synthetic or semi-synthetic libraries can be obtained from natural V_{HH} libraries by method such as random mutagenesis and/or CDR shuffling.

Camelized, Humanized or Human Antibodies

In an embodiment, the anti-BCMA single domain antibody is a natural Camelidae antibody or a chimeric antibody, such as a camelized antibody, a humanized antibody or a human antibody, more preferably a humanized antibody.

As used herein, “camelized” refers to the substitution of one or more amino acid residues from the amino acid sequence of a (naturally occurring) V_{H} domain of a conventional four-chain antibody by one or more amino acid residues present at one or more corresponding positions in the V_{HH} domain of a heavy chain antibody. This can be achieved by means known to those skilled in the art. Preferably, such “camelized” substitutions are inserted at amino acid positions forming and/or present at the VH-VL interface, and/or at so-called Camelidae residues (see e.g. WO1994004678, Riechmann and Muyldermans J. Immunol. Meth. 231:25-38, 1999).

As used herein, a “humanized” antibody refers to an antibody in which all or substantially all CDR amino acid residues are derived from non-human CDRs and all or substantially all FR amino acid residues are derived from human FRs. “Humanized forms” of a non-human antibody refer to variants of such non-human antibody that have been humanized to generally reduce immunogenicity in humans, while retaining the specificity and affinity of the parent non-human antibody. In some embodiments, some FR residues in a humanized antibody are substituted with corresponding residues from a non-human antibody (e.g., an antibody from which the CDR residues are derived), e.g., to restore or improve antibody specificity or affinity.

Humanized antibodies and methods for their preparation are well known to those skilled in the art, see e g Almagro and Fransson, Front. Biosci. 13:1619-1633 (2008). Human framework regions that can be used for humanization include, but are not limited to: framework regions selected using a “best fit” approach; framework regions derived from the consensus sequences of human antibodies of a particular subgroup of light or heavy chain variable regions; human mature (somatically mutated) framework regions or human germline framework regions; and framework regions obtained from screening FR libraries.

In some embodiments, the anti-BCMA single domain antibody is modified, e.g., humanized, without reducing its natural affinity for an antigen, while reducing its immunogenicity to a heterologous species. For example, the amino acid residues of the antibody variable domain (V_{HH}) of a llama antibody can be determined and, for example, one or more Camelidae amino acids in the framework regions are replaced by their human counterparts. Humanization does not significantly affect the antigen-binding ability of the resulting polypeptide. Humanization of Camelidae single domain antibodies requires mutagenesis of only a limited number of amino acids in a single polypeptide chain. This is in contrast to the humanization of scFv, Fab′, (Fab′)2 and IgG, which requires the introduction of amino acid changes in both light and heavy chains, and ensures the pairing capabilities of the two chains.

As used herein, the term “human antibody” refers to an antibody whose amino acid sequence corresponds to that of an antibody produced by a human or human cell, or using a human antibody library or a non-human source of other human antibody coding sequences, including human antibody libraries. Various techniques for producing human antibodies are known in the art. For example, human antibodies (e.g., human single domain antibodies) can be prepared by immunizing transgenic animals that have been genetically engineered to respond to antigenic challenge to produce fully human antibodies or fully antibodies with human variable regions. Such transgenic animals typically contain all or part of the human immunoglobulin loci, which replace the endogenous immunoglobulin loci, or which are present extrachromosomally or integrated randomly into the animal's chromosomes. In such transgenic animals (e.g., mice), endogenous immunoglobulin loci have typically been inactivated. For a review of methods for obtaining human antibodies from transgenic animals, see Lonberg, Nat. Biotech. 23:1117-1125 (2005). Transgenic mice or rats capable of producing fully human single domain antibodies are known in the art, see e.g. US20090307787A1, U.S. Pat. No. 8,754,287, US20150289489A1, US20100122358A1 and WO 2004049794.

In addition, human antibodies, such as human single domain antibodies, can also be produced by the hybridoma method or by isolating variable domain sequences of Fv clones selected from human phage display libraries.

Therefore, the present disclosure further provides a humanized anti-BCMA single domain antibody comprising FR1 selected from the group consisting of SEQ ID NO: 43, SEQ ID NO: 46, SEQ ID NO: 50, SEQ ID NO: 52, SEQ ID NO: 55, SEQ ID NO: 56, SEQ ID NO: 57, SEQ ID NO: 60, SEQ ID NO: 67 or variants thereof, 1-R2 selected from the group consisting of SEQ ID NO: 9, SEQ ID NO: 13, SEQ ID NO: 47, SEQ ID NO: 61 or variants thereof, FR3 selected from the group consisting of SEQ ID NO: 44, SEQ ID NO: 48, SEQ ID NO: 51, SEQ ID NO: 53, SEQ ID NO: 54, SEQ ID NO: 58, SEQ ID NO: 62, SEQ ID NO: 65, SEQ ID NO: 66, SEQ ID NO: 68, SEQ ID NO: 69 or variants thereof, and FR4 selected from the group consisting of SEQ ID NO: 15, SEQ ID NO: 24, SEQ ID NO: 45, SEQ ID NO: 49, SEQ ID NO: 59, SEQ ID NO: 63, SEQ ID NO: 64, SEQ ID NO: 70, SEQ ID NO: 71 or variants thereof, wherein the variant comprises substitution of at most 3 amino acids in the FR.

In an embodiment, the humanized anti-BCMA single domain antibody comprises:
(1) FR1 as set forth in SEQ ID NO: 43, SEQ ID NO: 52, SEQ ID NO: 55 or SEQ ID NO: 56, FR2 as set forth in SEQ ID NO: 13, FR3 as set forth in SEQ ID NO: 44, SEQ ID NO: 51, SEQ ID NO: 53 or SEQ ID NO: 54, FR4 as set forth in SEQ ID NO: 15 or SEQ ID NO: 45, or variants thereof, wherein the variant comprises substitution of at most 3 amino acids in the FR;
(2) FR1 as set forth in SEQ ID NO: 46 or SEQ ID NO: 50, FR2 as set forth in SEQ ID NO: 47, FR3 as set forth in SEQ ID NO: 48 or SEQ ID NO: 51, FR4 as set forth in SEQ ID NO: 49, or variants thereof, wherein the variant comprises substitution of at most 3 amino acids in the FR;
(3) FR1 as set forth in SEQ ID NO: 57 or SEQ ID NO: 67, FR2 as set forth in SEQ ID NO: 9, FR3 as set forth in SEQ ID NO: 58, SEQ ID NO: 65, SEQ ID NO: 66, SEQ ID NO: 68 or SEQ ID NO: 69, FR4 as set forth in SEQ ID NO: 24, SEQ ID NO: 59, SEQ ID NO: 63, SEQ ID NO: 64, SEQ ID NO: 70 or SEQ ID NO: 71, or variants thereof, wherein the variant comprises substitution of at most 3 amino acids in the FR; or
(4) FR1 as set forth in SEQ ID NO: 60, FR2 as set forth in SEQ ID NO: 61, FR3 as set forth in SEQ ID NO: 62, FR4 as set forth in SEQ ID NO: 63, or variants thereof, wherein the variant comprises substitution of at most 3 amino acids in the FR.

More preferably, the humanized anti-BCMA single domain antibody has at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NO: 72-86. Preferably, the amino acid sequence of the anti-BCMA single domain antibody is set forth in SEQ ID NO: 72-86.

In an aspect, the present disclosure further provides a multispecific antibody (preferably a bispecific antibody or a trispecific antibody) comprising the anti-BCMA single domain antibody (including humanized single domain antibody) as described above, and one or more second antibodies or antigen-binding portions thereof that specifically bind to antigens different from BCMA.

As used herein, the term “multispecific” means that the antigen binding protein has polyepitopic specificity (i.e., is capable of specifically binding two, three or more different epitopes on one biomolecule or is capable of specifically binding binds epitopes on two, three or more different biomolecules). As used herein, the term “bispecific” means that an antigen binding protein has two different antigen binding specificities.

In an embodiment, the second antibody or antigen-binding portion thereof may be in the form of any antibody or antibody fragment, such as a full-length antibody, Fab, Fab′, (Fab′)₂, Fv, scFv, scFv-scFv, a minibody, a diabody or sdAb.

Thus, in an embodiment, the second antibody, or antigen binding portion thereof, targets an antigen selected from the group consisting of: CD4, CD5, CD7, CD8, CD14, CD15, CD19, CD20, CD21, CD22, CD23, CD25, CD33, CD37, CD38, CD40, CD40L, CD46, CD52, CD54, CD74, CD80, CD126, CD138, B7, MUC-1, Ia, HM1.24, HLA-DR, tenascin, angiogenic factor, VEGF, PIGF, ED-B fibronectin, oncogene, oncogene product, CD66a-d, necrosis antigen, Ii, IL-2, T101, TAC, IL-6, ROR1, TRAIL-R1 (DR4), TRAIL-R2 (DR5), tEGFR, Her2, L1-CAM, mesothelin, CEA, hepatitis B surface antigen, antifolate receptor, CD24, CD30, CD44, EGFR, EGP-2, EGP-4, EPHa2, ErbB2, ErbB3, ErbB4, erbB dimer, EGFR vIII, FBP, FCRL5, FCRH5, fetal acetylcholine receptor, GD2, GD3, G protein-coupled receptor type C family 5D (GPRC5D), HMW-MAA, IL-22R-α, IL-13R-α2, kdr, κ light chain, Lewis Y, L1-cell adhesion molecule (L1-CAM), melanoma-associated antigen (MAGE)-A1, MAGE-A3, MAGE-A6, melanoma preferentially expressed antigen (PRAME), survivin, EGP2, EGP40, TAG72, B7-H6, IL-13 receptor a2 (IL-13Ra2), CA9, CD171, G250/CAIX, HLA-A1 MAGE A1, HLA-A2NY-ESO-1, PSCA, folate receptor-a, CD44v6, CD44v7/8, avb6 integrin, 8H9, NCAM, VEGF receptor, 5T4, fetal AchR, NKG2D ligand, dual antigens, antigens associated with common tags, cancer-testis antigen, MUC1, MUC16, NY-ESO-1, MART-1, gplOO, carcinoembryonic antigen, VEGF-R2, carcinoembryonic antigen (CEA), prostate specific antigen, PSMA, Her2/neu, estrogen receptor, progesterone receptor, ephrin B2, CD123, c-Met, GD-2, 0-acetylated GD2 (OGD2), CE7, Wilms tumor 1 (WT-1), cyclin, cyclin A2, CCL-1, hTERT, MDM2, CYP1B, WT1, survivin, AFP, p53, cyclin (D1), CS-1, BAFF-R, TACI, CD56, TIM-3, CD123, L1-cell adhesion molecules, MAGE-A1, MAGEA3, cell cyclins (e.g., cyclin A1 (CCNA1)) and/or pathogen-specific antigens, biotinylated molecules, molecules expressed by HIV, HCV, HBV and/or other pathogens; and/or neo-epitopes or neoantigens.

Nucleic Acid, Vector, Host Cell

In another aspect, the present disclosure relates to a nucleic acid molecule encoding the anti-BCMA single domain antibody or multispecific antibody of the present disclosure. The nucleic acid of the present disclosure may be RNA, DNA or cDNA. According to an embodiment of the present disclosure, the nucleic acid of the present disclosure is a substantially isolated nucleic acid.

In an embodiment, the nucleic acid molecule encoding the anti-BCMA single domain antibody has at least 90%, at least 91%, at least 92%, at least 93%, at least 94%, at least 95%, at least 96%, at least 97%, at least 98%, at least 99%, or 100% sequence identity to a nucleotide sequence selected from the group consisting of SEQ ID NO: 34-42 and 87-101, and can specifically bind to BCMA antigen. Preferably, the nucleic acid molecule encoding the anti-BCMA single domain antibody is set forth in SEQ ID NO: 34-42 and 87-101.

The nucleic acid of the present disclosure may also be in the form of, may be present in and/or may be part of a vector, such as a plasmid, cosmid or YAC. The vector may especially be an expression vector, i.e., a vector providing for expression of the anti-BCMA single domain antibody in vitro and/or in vivo (i.e., in a suitable host cell, host organism and/or expression system). The expression vector typically comprises at least one nucleic acid molecule of the present disclosure operably linked to one or more suitable expression regulatory elements (e.g., promoter, enhancer, terminator, etc.). Selection of such regulatory elements and their sequences for expression in a particular host is well known to those skilled in the art. Specific examples of regulatory elements and other elements useful or necessary for expression of the BCMA single domain antibodies of the present disclosure include, but are not limited to, promoter, enhancer, terminator, integrator, selectable marker, leader sequence, reporter gene.

In another aspect, the present disclosure further provides a host cell expressing the anti-BCMA single domain antibody or the multispecific antibody of the present disclosure and/or a host cell containing the nucleic acid or vector of the present disclosure. Preferred host cells of the present disclosure are bacterial cells, fungal cells or mammalian cells.

Suitable bacterial cells include cells of Gram-negative bacterial strains (e.g., Escherichia coli strains, Proteus strains, and Pseudomonas strains) and Gram-positive bacterial strains (e.g., Bacillus strains, Streptomyces strains, Staphylococcus strains and Lactococcus strains).

Suitable fungal cells include cells of species of Trichoderma, Neurospora, and Aspergillus; or cells of species of Saccharomyces (e.g., Saccharomyces cerevisiae), Schizosaccharomyces (e.g., Schizosaccharomyces pombe), Pichia (e.g., Pichia pastoris and Pichia methanolica) and Hansenula.

Suitable mammalian cells include, for example, HEK293 cells, CHO cells, BHK cells, HeLa cells, COS cells, and the like.

However, amphibian cells, insect cells, plant cells, and any other cells known in the art for expressing heterologous proteins can also be used in the present disclosure.

Chimeric Antigen Receptor

In another aspect, the present disclosure further provides a recombinant receptor, such as a recombinant TCR receptor or a chimeric antigen receptor comprising the anti-BCMA single domain antibody as described above. Preferably, the present disclosure further provides a chimeric antigen receptor comprising the anti-BCMA single domain antibody as described above.

As used herein, the term “chimeric antigen receptor” or “CAR” refers to an artificially constructed hybrid polypeptide that generally includes a ligand-binding domain (e.g., an antigen-binding portion of an antibody), a transmembrane domain, an optional co-stimulatory domain, and an intracellular signaling domain, which domains being connected by linkers. CARs are able to redirect the specificity and reactivity of T cells and other immune cells to a selected target in a non-MHC-restricted manner through the antigen-binding properties of antibodies.

In an embodiment, the present disclosure provides a chimeric antigen receptor comprising the anti-BCMA single domain antibody (including humanized single domain antibody) or the multispecific antibody comprising the anti-BCMA single domain antibody, a transmembrane domain and an intracellular signaling domain.

As used herein, the term “transmembrane domain” refers to a polypeptide structure that enables expression of a chimeric antigen receptor on the surface of an immune cell (e.g., a lymphocyte, an NK cell, or an NKT cell), and guides a cellular response of the immune cell against the target cell. The transmembrane domain may be natural or synthetic, and also may be derived from any membrane-bound protein or transmembrane protein. The transmembrane domain is capable of signaling when the chimeric antigen receptor binds to the target antigen. The transmembrane domains particularly suitable for use in the present disclosure may be derived from, for example, a TCRα chain, a TCRβ chain, a TCRγ chain, a TCRδ chain, a CD3ζ subunit, a CD3ε subunit, a CD3γ subunit, a CD3δ subunit, CD45, CD4, CD5, CD8α, CD9, CD16, CD22, CD33, CD28, CD37, CD64, CD80, CD86, CD134, CD137, CD154, and functional fragments thereof. Alternatively, the transmembrane domain may be synthesized and may mainly contain hydrophobic residues such as leucine and valine. Preferably, the transmembrane domain is derived from CD8a or CD28, and has at least 70%, preferably at least 80%, more preferably at least 90%, at least 95%, at least 97% or at least 99% or 100% sequence identity to an amino acid sequence as set forth in SEQ ID NO: 107 or SEQ ID NO: 114, or the encoding sequence thereof has at least 70%, preferably at least 80%, more preferably at least 90%, at least 95%, at least 97% or at least 99% or 100% sequence identity to a nucleic acid molecule as set forth in SEQ ID NO: 106 or SEQ ID NO: 115.

In an embodiment, the chimeric antigen receptors of the present disclosure may further comprise a hinge region located between the antibody and the transmembrane domain. As used herein, the term “hinge region” generally refers to any oligopeptide or polypeptide that functions to link a transmembrane domain to a ligand binding domain Specifically, the hinge region serves to provide greater flexibility and accessibility to the ligand binding domain. The hinge region may comprise up to 300 amino acids, preferably 10 to 100 amino acids and most preferably 25 to 50 amino acids. The hinge region may be completely or partially derived from a natural molecule, for example, completely or partially from the extracellular region of CD8, CD4 or CD28, or completely or partially from an antibody constant region. Alternatively, the hinge region may be a synthetic sequence corresponding to a naturally occurring hinge sequence, or may be a completely synthetic hinge sequence. In a preferred embodiment, the hinge region comprises a hinge region portion of CD8α, CD28, an Fc γ RIII α receptor, IgG4, or IgG1, more preferably a hinge from CD8α, CD28 or IgG4, and has at least 70%, preferably at least 80%, more preferably at least 90%, at least 95%, at least 97% or at least 99% or 100% sequence identity to an amino acid sequence as set forth in SEQ ID NO: 105, 120 or 122, or the encoding sequence thereof has at least 70%, preferably at least 80%, more preferably at least 90%, at least 95%, at least 97% or at least 99% or at least 100% sequence identity to a nucleic acid molecule as set forth in SEQ ID NO: 104, 121 or 123.

As used herein, the term “intracellular signaling domain” refers to a protein portion that transduces an effector function signal and guides a cell to perform a specified function. In an embodiment, the intracellular signaling domain contained in the chimeric antigen receptor of the present disclosure may be intracellular sequences of a T cell receptor and a co-receptor, upon binding of antigen receptor, which act together to initiate signaling, as well as any derivative or variant of these sequences and any synthetic sequence having the same or similar function. The intracellular signaling domain may contain many immunoreceptor tyrosine-based activation motifs (ITAM). Non-limiting examples of intracellular signaling domain of the present disclosure include, but are not limited to, intracellular regions of FcRγ, FcRβ, CD3γ, CD3δ, CD3ε, CD3ζ, CD22, CD79a, CD79b, and CD66d. In a preferred embodiment, the signaling domain of the CAR of the present disclosure may contain a CD3ζ intracellular region, which has at least 70%, preferably at least 80%, more preferably at least 90%, at least 95%, at least 97%, or at least 99% or 100% sequence identity to an amino acid sequence as set forth in SEQ ID NO: 111 or SEQ ID NO: 116, or the encoding sequence thereof has at least 70%, preferably at least 80%, more preferably at least at least 90%, at least 95%, at least 97%, or at least 99% or 100% sequence identity to a nucleic acid molecule as set forth in SEQ ID NO: 110 or SEQ ID NO: 117.

In an embodiment, the chimeric antigen receptor may also comprise one or more co-stimulatory domains. The co-stimulatory domain may be an intracellular functional signaling domain from a co-stimulatory molecule, which comprises the entire intracellular portion of the co-stimulatory molecule, or a functional fragment thereof. A “costimulatory molecule” refers to a cognate binding partner that specifically binds to a costimulatory ligand on a T cell, thereby mediating a costimulatory response (e.g., proliferation) of the T cell. Costimulatory molecules include, but are not limited to, MHC class 1 molecules, BTLA, and Toll ligand receptors. Non-limiting examples of costimulatory domains of the present disclosure include, but are not limited to, costimulatory signaling domains derived from the following proteins: TLR1, TLR2, TLR3, TLR4, TLR5, TLR6, TLR7, TLR8, TLR9, TLR10, CARD11, CD2, CD7, CD8, CD18 (LFA-1), CD27, CD28, CD30, CD40, CD54 (ICAM), CD83, CD134 (OX40), CD137 (4-1BB), CD270 (HVEM), CD272 (BTLA), CD276 (B7-H3), CD278 (ICOS), CD357 (GITR), DAP10, LAT, NKG2C, SLP76, PD-1, LIGHT, TRIM, and ZAP70. Preferably, the costimulatory domain of the CAR of the present disclosure is from 4-1BB, CD28 or 4-1BB+CD28. In an embodiment, the 4-1BB co-stimulatory domain has at least 70%, preferably at least 80%, more preferably at least 90%, at least 95%, at least 97% or at least 99% or 100% sequence identity to an amino acid sequence as set forth in SEQ ID NO: 109, or the coding sequence thereof has at least 70%, preferably at least 80%, more preferably at least 90%, at least 95%, at least 97% or at least 99% or 100% sequence identity to the nucleic acid molecule as set forth in SEQ ID NO: 108. In an embodiment, the CD28 co-stimulatory domain has at least 70%, preferably at least 80%, more preferably at least 90%, at least 95%, at least 97% or at least 99% or 100% sequence identity to an amino acid sequence as set forth in SEQ ID NO: 113, or the coding sequence thereof has at least 70%, preferably at least 80%, more preferably at least 90%, at least 95%, at least 97% or at least 99% or 100% sequence identity to the nucleic acid molecule as set forth in SEQ ID NO: 112.

In an embodiment, the CAR of the present disclosure may further comprise a signal peptide such that when it is expressed in a cell such as a T cell, the nascent protein is directed to the endoplasmic reticulum and subsequently to the cell surface. The core of the signal peptide may contain a long hydrophobic amino acid segment, which has a tendency to form a single α-helix. At the end of the signal peptide, there is usually an amino acid segment capable of being recognized and cleaved by signal peptidase. The signal peptidase can cleave during or after translocation, so as to generate free signal peptide and mature protein. Then, the free signal peptide is digested by a specific protease. Signal peptides that can be used in the present disclosure are well known to those skilled in the art, for example, signal peptides derived from B2M, CD8α, IgG1, GM-CSFRα, and the like. In an embodiment, the signal peptide that can be used in the present disclosure is from CD8α or B2M, and has at least 70%, preferably at least 80%, more preferably at least at least 90%, at least 95%, at least 97% or at least 99% or 100% sequence identity to an amino acid sequence as set forth in SEQ ID NO: 103 or SEQ ID NO: 108, or the coding sequence thereof has at least 70%, preferably at least 80%, more preferably at least 90%, at least 95%, at least 97% or at least 99% or 100% sequence identity to the nucleic acid molecule as set forth in SEQ ID NO: 102 or SEQ ID NO: 119.

In an embodiment, the CAR comprises the anti-BCMA single domain antibody (including humanized single domain antibody) or the multispecific antibody comprising the anti-BCMA single domain antibody as provided herein, a CD8α transmembrane region, a CD28 or 4-1BB co-stimulatory domain, and a CD3ζ intracellular signaling domain. In this embodiment, the CAR may further comprise a signal peptide from B2M, CD8α, IgG1 or GM-CSFRα.

The present disclosure further provides a nucleic acid molecule encoding the BCMA-targeting chimeric antigen receptor as defined above, and a vector comprising the nucleic acid molecule.

As used herein, the term “vector” is an intermediary nucleic acid molecule used to transfer (exogenous) genetic material into a host cell, and in the host cell the nucleic acid molecule can be, for example, replicated and/or expressed. The vector generally includes targeting vectors and expression vectors. The “targeting vector” is a medium that delivers an isolated nucleic acid to the interior of a cell by, for example, homologous recombination or by using a hybrid recombinase of a sequence at specific target site. The “expression vector” is a vector used for transcription of heterologous nucleic acid sequences (for example, those sequences encoding the chimeric antigen receptor polypeptides of the present disclosure) in suitable host cells and the translation of their mRNAs. Suitable vectors that can be used in the present disclosure are known in the art, and many are commercially available. In an embodiment, the vector of the present disclosure includes, but is not limited to, plasmid, virus (e.g., retrovirus, lentivirus, adenovirus, vaccinia virus, Rous sarcoma virus (RSV), polyoma virus, and adeno-associated virus (AAV), etc.), phage, phagemid, cosmid, and artificial chromosome (including BAC and YAC). The vector itself is usually a nucleic acid molecule, and usually is a DNA sequence containing an insert (transgene) and a larger sequence as “backbone” of the vector. Engineered vector typically also contains an origin autonomously replicating in the host cell (if stable expression of polynucleotide is desired), a selectable marker, and a restriction enzyme cleavage site (e.g., a multiple cloning site, MCS). The vectors may additionally contain elements such as a promoter, a poly-A tail (polyA), 3′ UTR, an enhancer, a terminator, an insulator, an operon, a selectable marker, a reporter gene, a targeting sequence, and/or a protein purification tag. In a specific embodiment, the vector is an in vitro transcription vector.

Engineered Immune Cells

In an aspect, the present disclosure further provides an engineered immune cell expressing the CAR of the present disclosure.

As used herein, the term “immune cell” refers to any cell of the immune system that has one or more effector functions (e.g., cytotoxic cell killing activity, secretion of cytokines, induction of ADCC and/or CDC). For example, the immune cell may be a T cell, a macrophage, a dendritic cell, a monocyte, an NK cell, and/or an NKT cell. In an embodiment, the immune cell is derived from a stem cell, such as an adult stem cell, an embryonic stem cell, a cord blood stem cell, a progenitor cell, a bone marrow stem cell, an induced pluripotent stem cell, a totipotent stem cell, or a hematopoietic stem cell, and so on. Preferably, the immune cell is a T cell. The T cell may be any T cell, such as in vitro cultured T cell, for example, primary T cell, or T cell from in vitro cultured T cell line, e.g., Jurkat, SupT1, etc., or T cell obtained from a subject. Examples of subject include humans, dogs, cats, mice, rats, and transgenic species thereof. The T cell can be obtained from a variety of sources, including peripheral blood monocytes, bone marrow, lymph node tissue, umbilical blood, thymus tissue, tissue from sites of infection, ascites, pleural effusion, spleen tissue, and tumors. The T cell also may be concentrated or purified. The T cell may be at any stage of development including, but not limited to, a CD4+/CD8+ T cell, a CD4+ helper T cell (e.g., Th1 and Th2 cells), CD8+ T cell (e.g., cytotoxic T cell), tumor infiltrating cell, memory T cell, naive T cell, γδ-T cell, αβ-T cell. In a preferred embodiment, the immune cell is a human T cell. The T cell can be isolated from the blood of a subject using a variety of techniques known to those of skill in the art, such as Ficoll.

The nucleic acid sequence encoding the chimeric antigen receptor can be introduced into an immune cell using conventional methods known in the art (e.g., by transduction, transfection, transformation). “Transfection” is a process of introducing a nucleic acid molecule or polynucleotide (including a vector) into a target cell. An example is RNA transfection, i.e., the process of introducing RNA (e.g., in vitro transcribed RNA, ivtRNA) into a host cell. This term is mainly used for a non-viral method in eukaryotic cells. The term “transduction” is generally used to describe virus-mediated transfer of nucleic acid molecules or polynucleotides. Transfection of animal cells typically involves opening transient pores or “holes” in the cell membrane, so as to allow uptake of material. Transfection may be carried out using calcium phosphate, by electroporation, by extrusion of cells, or by mixing cationic lipids with the material so as to produce liposomes which fuse with the cell membrane and deposit their cargo into the interior. Exemplary techniques for transfecting eukaryotic host cells include lipid vesicle-mediated uptake, heat shock-mediated uptake, calcium phosphate-mediated transfection (calcium phosphate/DNA co-precipitation), microinjection, and electroporation. The term “transformation” is used to describe the non-virus transfer of a nucleic acid molecule or polynucleotide (including a vector) to bacteria, and also to non-animal eukaryotic cells (including plant cells). Thus, the transformation is a genetic alteration of bacterial or non-animal eukaryotic cells, which is produced by direct uptake of a cell membrane from its surroundings and subsequent incorporation of exogenous genetic material (nucleic acid molecule). The transformation can be achieved by artificial means. In order for transformation to occur, the cell or bacterium must be in a competent state. For prokaryotic transformation, the techniques may include heat shock-mediated uptake, fusion to bacterial protoplasts of intact cells, microinjection, and electroporation. After the nucleic acid or vector is introduced into the immune cells, those skilled in the art can amplify and activate the obtained immune cells by conventional techniques.

In an embodiment, in order to reduce the risk of graft-versus-host disease, the engineered immune cell further comprises suppressed or silenced expression of at least one gene selected from the group consisting of: CD52, GR, dCK, TCR/CD3 genes (e.g., TRAC, TRBC, CD3γ, CD3δ, CD3ε, CD3ζ), MHC related genes (HLA-A, HLA-B, HLA-C, B2M, HLA-DPA, HLA-DQ, HLA-DRA, TAP1, TAP2, LMP2, LMP7, RFX5, RFXAP, RFXANK, CIITA) and immune checkpoint genes such as PD1, LAG3, TIM3, CTLA4, PPP2CA, PPP2CB, PTPN6, PTPN22, PDCD1, HAVCR2, BTLA, CD160, TIGIT, CD96, CRTAM, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT, FOXP3, PRDM1, BATF, GUCY1A2, GUCY1A3, GUCY1B2 and GUCY1B3. Preferably, the engineered immune cell further comprises suppressed or silenced expression of at least one gene selected from the group consisting of: TRAC, TRBC, HLA-A, HLA-B, HLA-C, B2M, RFX5, RFXAP, RFXANK, CIITA, PD1, LAG3, TIM3, CTLA4, more preferably TRAC, TRBC, HLA-A, HLA-B, HLA-C, B2M, RFX5, RFXAP, RFXANK, CIITA.

Methods of inhibiting gene expression or silencing genes are well known to those skilled in the art. For example, antisense RNA, RNA decoys, RNA aptamers, siRNA, shRNA/miRNA, trans dominant negative protein (TNP), chimeric/antibody conjugates, chemokine ligands, anti-infective cellular proteins, intracellular antibodies (sFv), nucleoside analogs (NRTI), non-nucleoside analogs (NNRTI), integrase inhibitors (oligonucleotides, dinucleotides, and chemical agents), and protease inhibitors may be used to inhibit the expression of genes. Alternatively, genes can also be silenced by DNA fragmentation mediated by for example meganucleases, zinc finger nucleases, TALE nucleases or Cas enzymes in CRISPR systems.

In an embodiment, a plurality of immune cells is provided, each immune cell engineered to express one or more chimeric antigen receptors. For example, in some embodiments, an immune cell is engineered to express a chimeric antigen receptor that binds and/or targets BCMA (e.g., a CAR comprising the anti-BCMA single domain antibody of the present disclosure), and another cell is engineered to express a chimeric antigen receptor that binds and/or targets antigens different from BCMA. In an embodiment, immune cells may also express multispecific chimeric antigen receptors that target one or more antigens, including BCMA. For example, such a multispecific chimeric antigen receptor may comprise a multispecific antibody targeting BCMA, or comprise both the anti-BCMA single domain antibody of the present disclosure and antibodies targeting antigens different from BCMA. In such embodiments, the plurality of engineered immune cells may be administered together or separately. In an embodiment, the plurality of immune cells can be in the same composition or in different compositions. Exemplary compositions of cells include those described in the following sections of this application.

Antibody Conjugate

In an aspect, the present disclosure provides an antibody conjugate comprising the anti-BCMA single domain antibody as defined in the present disclosure and a second functional structure, wherein the second functional structure is selected from the group consisting of Fc, a radioisotope, a structure moiety for extending half-life, a detectable marker and a drug.

In an embodiment, the present disclosure provides an antibody conjugate comprising the anti-BCMA single domain antibody as defined in the present disclosure and Fc. As used herein, the term “Fc” is used to define the C-terminal region of an immunoglobulin heavy chain, and includes natural Fc and variant Fc. “Natural Fc” refers to a molecule or sequence comprising a non-antigen-binding fragment, in a monomeric or multimeric form, produced by digestion of an intact antibody. The source of immunoglobulin from which natural Fc is produced is preferably of human origin. Natural Fc fragments are composed of monomeric polypeptides that can be linked as dimers or multimers through covalent linkages (e.g., disulfide bonds) and non-covalent linkages. Depending on the class (e.g., IgG, IgA, IgE, IgD, IgM) or subtype (e.g., IgG1, IgG2, IgG3, IgA1, IgGA2), natural Fc molecules have 1-4 intermolecular disulfide bonds between monomeric subunits. An example of a natural Fc is a disulfide-linked dimer produced by papain digestion of IgG (see Ellison et al. (1982), Nucleic Acids Res. 10:4071-9). The term “natural Fc” as used herein generally refers to monomeric, dimeric and multimeric forms. A “variant Fc” refers to an amino acid sequence that differs from that of a “natural” or “wild-type” Fc by virtue of at least one “amino acid modification” as defined herein, also referred to as an “Fc variant”. Thus, “Fc” also includes single-chain Fc (scFc), i.e., a single-chain Fc consisting of two Fc monomers linked by a polypeptide linker, which is capable of naturally folding into a functional dimeric Fc region. In an embodiment, the Fc is preferably the Fc of a human immunoglobulin, more preferably the Fc of a human IgG1.

In an embodiment, the present disclosure provides an antibody conjugate comprising the anti-BCMA single domain antibody as defined in the present disclosure and a radioactive isotope. Examples of radioisotopes useful in the present disclosure include, but are not limited to, At²¹¹, I¹³¹, I¹²⁵, Y⁹⁰, Re¹⁸⁶, Re¹⁸⁸, Sm¹⁵³, Bi²¹², P³², Pb²¹², ^{99m}Tc, ¹²³I, ¹⁸F, and ⁶⁸Ga.

In an embodiment, the present disclosure provides an antibody conjugate comprising the anti-BCMA single domain antibody as defined in the present disclosure and a structure moiety for extending half-life selected from the group consisting of an albumin-binding structure, a transferrin-binding structure, a polyethylene glycol molecule, a recombinant polyethylene glycol molecule, human serum albumin, a fragment of human serum albumin, and a polypeptide binding to human serum albumin (including antibody).

In an embodiment, the present disclosure provides an antibody conjugate comprising the anti-BCMA single domain antibody as defined in the present disclosure and a detectable marker. The term “detectable marker” means herein a compound that produces a detectable signal. For example, the detectable marker may be an MRI contrast agent, a scintigraphy contrast agent, an X-ray imaging contrast agent, an ultrasound contrast agent, an optical imaging contrast agent. Examples of detectable markers include fluorophores (e.g., fluorescein, Alexa, or cyanine), chemiluminescent compounds (e.g., luminol), bioluminescent compounds (e.g., luciferase or alkaline phosphatase), enzymes (e.g., horseradish peroxidase, glucose-6-phosphatase, β-galactosidase), antibiotics (e.g., kanamycin, ampicillin, chloramphenicol, tetracycline, etc.) resistance genes, and contrast agents (e.g., nanoparticles or gadolinium). Those skilled in the art can select an appropriate detectable marker according to the detection system used.

In an embodiment, the present disclosure provides an antibody conjugate comprising the anti-BCMA single domain antibody as defined in the present disclosure and a drug conjugated to the anti-BCMA single domain antibody, such as a cytotoxin or an immunomodulator (i.e., an antibody-drug conjugate). Usually, the drug is covalently linked to the antibody, usually by a linker. In an embodiment, the drug is a cytotoxin. In another embodiment, the drug is an immunomodulator. Examples of cytotoxins include, but are not limited to, methotrexate, aminopterin, 6-mercaptopurine, 6-thioguanine, cytarabine, 5-fluorouracil, dacarbazine, nitrogen mustard, thiotepa, chlorambucil, melphalan, carmustine (BSNU), lomustine (CCNU), 1-methylnitrosourea, cyclophosphamide, nitrogen mustard, busulfan, dibromomannitol, streptozocin, mitomycin, cis-dichlorodiamine platinum (II) (DDP), cisplatin, carboplatin, zorubicin, doxorubicin, detorubicin, carminomicin, idarubicin, epirubicin, mitoxantrone, actinomycin D, bleomycin, calicheamicin, mithramycin, antramycin (AMC), vincristine, vinblastine, paclitaxel, ricin, pseudomonas exotoxin, gemcitabine, cytochalasin B, gramicidin D, ethidium bromide, emetine, etoposide, teniposide, colchicine, mitoxantrone, 1-dehydrotestosterone, glucocorticoid, procaine, tetracaine, lidocaine, propranolol, puromycin, procarbazine, hydroxyurea, asparaginase, corticosteroids, mitotane (O,P′-(DDD)), interferon, and combinations thereof. Examples of immunomodulators include, but are not limited to, ganciclovir, etanercept, tacrolimus, sirolimus, voclosporin, cyclosporine, rapamycin, cyclophosphamide, azathioprine, mycophenolate mofetil, methotrexate, glucocorticoid and analogs thereof, cytokines, stem cell growth factors, lymphotoxins, tumor necrosis factor (TNF), hematopoietic factors, interleukins (e.g., IL-1, IL-2, IL-3, IL-6, IL-10, IL-12, IL-18 and IL-21), colony-stimulating factors (e.g., G-CSF and (GM-CSF), interferons (e.g., interferon-α, interferon-beta and interferon-gamma), stem cell growth factor designated “S1 factor”, erythropoietin and thrombopoietin, or combinations thereof.

### Kits and Pharmaceutical Compositions

In another aspect, the present disclosure further provides a detection kit comprising the single domain antibody, the multispecific antibody, the antibody conjugate or the chimeric antigen receptor described in the present disclosure.

In another aspect, the present disclosure further provides a pharmaceutical composition comprising the single domain antibody, the chimeric antigen receptor, the multispecific antibody or the antibody conjugate of the present disclosure, and one or more pharmaceutically acceptable excipients.

As used herein, the term “pharmaceutically acceptable excipient” refers to a vector and/or excipient that is pharmacologically and/or physiologically compatible (i.e., capable of triggering a desired therapeutic effect without causing any undesired local or systemic effects) with the subject and active ingredient, and it is well known in the art (see, e.g., Remington's Pharmaceutical Sciences. Edited by Gennaro A R, 19^{th} ed. Pennsylvania: Mack Publishing Company, 1995). Examples of pharmaceutically acceptable excipient include, but are not limited to, filler, binder, disintegrant, coating agent, adsorbent, anti-adherent, glidant, antioxidant, flavoring agent, colorant, sweetener, solvent, co-solvent, buffer agent, chelating agent, surfactant, diluent, wetting agent, preservative, emulsifier, cladding agent, isotonic agent, absorption delaying agent, stabilizer, and tension regulator. It is known to those skilled in the art to select a suitable excipient to prepare the desired pharmaceutical composition of the present disclosure. Exemplary excipients for use in the pharmaceutical composition of the present disclosure include saline, buffered saline, dextrose, and water. Generally, the selection of a suitable excipient depends, in particular, on the active agent used, the disease to be treated, and the desired dosage form of the pharmaceutical composition.

The pharmaceutical composition according to the present disclosure is suitable for multiple routes of administration. Generally, the administration is parenterally accomplished. Parenteral delivery methods comprise topical, intraarterial, intramuscular, subcutaneous, intramedullary, intrathecal, intraventricular, intravenous, intraperitoneal, intrauterine, intravaginal, sublingual, or intranasal administration.

The pharmaceutical composition according to the present disclosure also can be prepared in various forms, such as solid, liquid, gaseous or lyophilized forms, particularly the pharmaceutical composition can be prepared in the form of ointment, cream, transdermal patch, gel, powder, tablet, solution, aerosol, granule, pill, suspension, emulsion, capsule, syrup, elixir, extract, tincture or liquid extract, or in a form particularly suitable for the desired method of administration. Processes known in the present disclosure for producing a medicine may include, for example, conventional mixing, dissolving, granulating, dragee-making, grinding, emulsifying, encapsulating, embedding or lyophilizing process. The pharmaceutical composition containing, for example, the immune cell as described herein is generally provided in a form of solution, and preferably contains a pharmaceutically acceptable buffer agent.

The pharmaceutical composition according to the present disclosure further may be administered in combination with one or more other agents suitable for the treatment and/or prophylaxis of diseases to be treated. Preferred examples of agent suitable for the combination include known anti-cancer medicines such as cisplatin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E, vincristine and doxorubicin; peptide cytotoxins, such as ricin, diphtheria toxin, pseudomonas exotoxin A, Dnase and Rnase; radionuclides such as iodine 131, rhenium 186, indium 111, iridium 90, bismuth 210, bismuth 213, actinides 225 and astatine 213; prodrugs such as antibody-directed enzyme prodrugs; immunostimulatory agents such as platelet factor 4, and melanoma growth stimulating protein; antibodies or fragments thereof, such as anti-CD3 antibodies or fragments thereof, complement activators, heterologous protein domains, homologous protein domains, viral/bacterial protein domains and viral/bacterial peptides. In addition, the pharmaceutical composition of the present disclosure also can be used in combination with one or more other treatment methods, such as chemotherapy and radiotherapy.

Therapeutic/Preventive/Diagnostic Use

In another aspect, the present disclosure further provides a method for treating and/or preventing and/or diagnosing diseases associated with BCMA expression, comprising administering to a subject the single domain antibody, the chimeric antigen receptor, the multispecific antibody, the antibody conjugate or the pharmaceutical composition as described above. Preferably, the disease associated with BCMA expression is selected from the group consisting of autoimmune diseases, lymphoma, leukemia or plasma cell malignancies.

In an embodiment, the disease associated with BCMA expression is an autoimmune disease, including but not limited to systemic lupus erythematosus (SLE), lupus nephritis, inflammatory bowel disease, rheumatoid arthritis (e.g., juvenile rheumatoid arthritis), ANCA-associated vasculitis, idiopathic thrombocytopenic purpura (ITP), thrombotic thrombocytopenic purpura (TTP), autoimmune thrombocytopenia, Chagas' disease, Grave's disease, Wegener's granulomatosis, polyarteritis nodosa, Sjogren's syndrome, pemphigus vulgaris, scleroderma, multiple sclerosis, psoriasis, IgA nephropathy, IgM multiple peripheral neuropathy, vasculitis, diabetes mellitus, Reynaud's syndrome, antiphospholipid syndrome, Goodpasture's disease, Kawasaki disease, autoimmune hemolytic anemia, myasthenia gravis, or progressive glomerulonephritis.

In an embodiment, the disease associated with BCMA expression is lymphoma, including but not limited to Burkitt's lymphoma (e.g., endemic Burkitt's lymphoma or sporadic Burkitt's lymphoma), non-Hodgkin's lymphoma (NHL), Hodgkin's lymphoma, Waldenstrom macroglobulinemia, follicular lymphoma, small non-cleaved cell lymphoma, mucosa-associated lymphoid tissue lymphoma (MALT), marginal zone lymphoma, splenic lymphoma, nodular monocytoid B-cell lymphoma, immunoblastic lymphoma, large cell lymphoma, diffuse mixed cell lymphoma, pulmonary B-cell angiocentric lymphoma, small lymphocyte lymphoma, primary mediastinal B-cell lymphoma, lymphoplasmacytic lymphoma (LPL), or mantle cell lymphoma (MCL).

In an embodiment, the disease associated with BCMA expression is leukemia, including but not limited to chronic lymphocytic leukemia (CLL), plasma cell leukemia or acute lymphoblastic leukemia (ALL).

In an embodiment, the disease associated with BCMA expression is a plasma cell malignancy, including but not limited to multiple myeloma (e.g., nonsecretory multiple myeloma, smoldering multiple myeloma) or plasmacytoma.

### BRIEF DESCRIPTION OF DRAWINGS

FIG. 1 shows the antibody expression levels in various BCMA CAR-T cells.

FIG. 2 shows the killing effect of various BCMA CAR-T cells on target cells K562-BCMA at different effector-to-target ratios.

FIG. 3 shows the IL2 release levels after co-culture of various BCMA CAR-T cells with target cells (K562-BCMA) and non-target cells (K562 and NUGC4).

FIG. 4 shows the IFN-γ release levels after co-culture of various BCMA CAR-T cells with target cells (K562-BCMA) and non-target cells (K562 and NUGC4).

FIG. 5 shows the degranulation of target cells (K562-BCMA) and non-target cells (K562 and NUGC4) by various BCMA CAR-T cells.

FIG. 6 shows the survival curves of mice treated with various BCMA CAR-T cells.

FIG. 7 shows the tumor burden in mice treated with various BCMA CAR-T cells as measured by in vivo optical imaging at different time points.

FIG. 8 shows the antibody expression level in BCMA CAR-T cells constructed with BH60 humanized single domain antibody.

FIG. 9 shows the antibody expression level in BCMA CAR-T cells constructed with BH86 humanized single domain antibody.

FIG. 10 shows the killing effect of humanized BCMA CAR-T cells on target cells MM.1S.

FIG. 11 shows the killing effect of humanized BCMA CAR-T cells on target cells K562-BCMA.

FIG. 12 shows the killing effect of humanized BCMA CAR-T cells on non-target cells K562.

FIG. 13 shows the IL2 release levels after co-culture of various humanized BCMA CAR-T cells with target cells (MM.1S and K562-BCMA) and non-target cells (K562, Jurkat, Nalm6, NUGC4 and 293T).

FIG. 14 shows the IFN-γ release levels after co-culture of various humanized BCMA CAR-T cells with target cells (MM.1S and K562-BCMA) and non-target cells (K562, Jurkat, Nalm6, NUGC4 and 293T).

FIG. 15 shows the tumor burden in mice treated with various humanized BCMA CAR-T cells as determined by in vivo optical imaging at different time points.

### EXAMPLE

### Example 1. Screening of Anti-BCMA Single Domain Antibody

Two llamas, numbered QLL217 and QL220 respectively, were immunized with BCMA protein (Acrobio systems, BCA-H522y), and then blood was collected on Jan. 2, Jan. 29, and Feb. 26, 2019, respectively. PBMCs were isolated from the blood samples, and a VHH single domain antibody phage library was constructed by methods known in the art. Specifically, the total RNA in PBMC was first extracted by the phenol-chloroform method, and then the total RNA was used as a template for reverse-transcribing into cDNA with a reverse transcription kit (Invitrogen) according to the instruction. VHH was amplified by nested PCR, and the target VHH fragment was identified by agarose gel electrophoresis and recovered. The recovered target VHH fragments were cloned into the phage display vector pADL20c, and then transformed into TG1 cells to construct an anti-BCMA single domain antibody library. The library capacity was determined to be 6.8×10⁹ by serial dilution plating.

Using methods known to those skilled in the art, clones specifically binding to BCMA were obtained from the constructed anti-BCMA single domain antibody library by ELISA through three rounds of screening. These clones were sequenced respectively, and the amino acid sequences were aligned to obtain 9 specific binding clones with different sequences, the amino acid sequences of which are shown in Table 1 below.

TABLE 1
anti-BCMA single domain antibody
clones and SEQ ID Nos thereof

| | | | | | | | | VHH | |
|---|---|---|---|---|---|---|---|---|---|
| | CDR | | | FR | | | | (SEQ | |
| | (SEQ ID NO) | | | (SEQ ID NO) | | | | ID NO) | |
| Clone | CDR1 | CDR2 | CDR3 | FR1 | FR2 | FR3 | FR4 | aa | nt |
| BH59 | 1 | 2 | 3 | 8 | 9 | 10 | 11 | 25 | 34 |
| BH60 | 4 | 5 | 6 | 12 | 13 | 14 | 15 | 26 | 35 |
| BH80 | 4 | 5 | 6 | 12 | 13 | 14 | 16 | 27 | 36 |
| BH81 | 7 | 5 | 6 | 17 | 18 | 19 | 15 | 28 | 37 |
| BH82 | 4 | 5 | 6 | 20 | 13 | 21 | 16 | 29 | 38 |
| BH83 | 4 | 5 | 6 | 20 | 13 | 21 | 15 | 30 | 39 |
| BH84 | 4 | 5 | 6 | 22 | 13 | 14 | 15 | 31 | 40 |
| BH86 | 1 | 2 | 3 | 8 | 9 | 23 | 24 | 32 | 41 |
| BH87 | 1 | 2 | 3 | 8 | 9 | 23 | 11 | 33 | 42 |

### Example 2. Construction of Chimeric Antigen Receptor Cells Targeting BCMA

Sequences encoding the following proteins were synthesized and cloned into pLVX vector (Public Protein/Plasmid Library (PPL), Cat. No.: PPL00157-4a): CD8α signal peptide (SEQ ID NO: 103), anti-BCMA single domain antibody (selected from any sequence of SEQ ID NO: 25-33), CD8α hinge region (SEQ ID NO: 105), CD8α transmembrane region (SEQ ID NO: 107), 4-1BB intracellular region (SEQ ID NO: 109) and CD3ζ intracellular region (SEQ ID NO: 111), and the correct insertion of the target sequence was confirmed by sequencing.

Three ml Opti-MEM (Gibco, Cat. No. 31985-070) was added to a sterile tube to dilute the above plasmid, and then packaging vector psPAX2 (Addgene, Cat. No. 12260) and envelope vector pMD2.G (Addgene, Cat. No. 12259) were added according to the ratio of plasmid:viralpackagingvector:viral envelope vector=4:2:1. Then, 120 μl X-treme GENE HP DNA transfection reagent (Roche, Cat. No. 06366236001) was added, mixed immediately, and incubated at room temperature for 15 min, and then the plasmid/vector/transfection reagent mixture was added dropwise to the culture flask of 293T cells. Viruses were collected at 24 hours and 48 hours, pooled, and ultracentrifuged (25000 g, 4° C., 2.5 hours) to obtain concentrated lentiviruses.

T cells were activated with DynaBeads CD3/CD28 CTSTM (Gibco, Cat. No. 40203D) and cultured at 37° C. and 5% CO₂ for 1 day. Then, the concentrated lentivirus was added, and after continuous culture for 3 days, BCMA CAR-T cells containing different BCMA single domain antibodies were obtained. Unmodified wild-type T cells (NT) were used as controls.

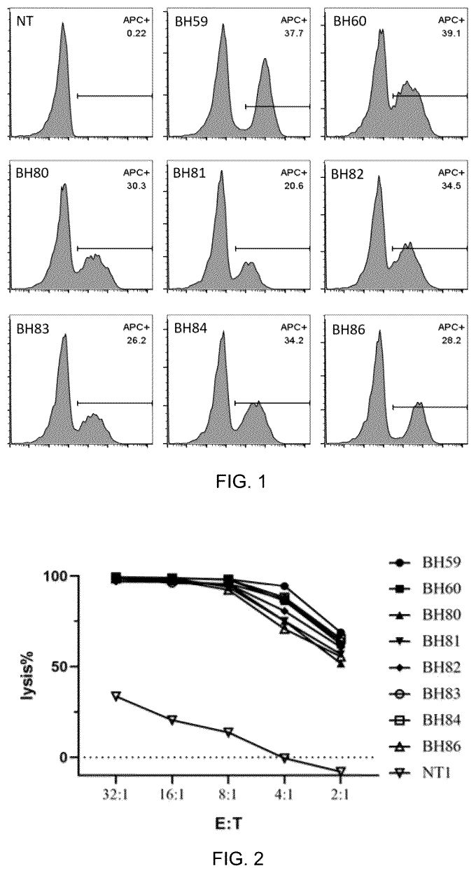

After culturing at 37° C. and 5% CO₂ for 11 days, the expression level of anti-BCMA single domain antibody on the BCMA CAR-T cells was detected by flow cytometry using MonoRab™ Rabbit Anti-Camelid VHH Antibody [Biotin], mAb (GenScript, Cat. No. A01995) as the primary antibody, and APC Streptavidin (BD Pharmingen, Cat. No. 554067) as the secondary antibody, and the results are shown in FIG. 1.

It can be seen that all the BCMA single domain antibodies in the CAR-T cells prepared by the present disclosure can be effectively expressed.

### Example 3. Killing Effect of BCMA-CAR T Cells on Target Cells and Release of Cytokines Thereof

3.1 Detection of the Ability to Kill Target Cells

First, K562-BCMA (gifted by Shenzhen Pregene Biopharma Co., Ltd) target cells carrying the fluorescein gene were plated into a 96-well plate at 1×10⁴/well, and then NT cells and CAR T cells were plated into the 96-well plate with effector-target ratios (i.e., the ratio of effector T cells to target cells) of 32:1, 16:1, 8:1, 4:1, and 2:1 for co-culture, and the fluorescence value was measured with a microplate reader after 16-18 hours. According to the calculation formula: (average fluorescence value of target cells—average fluorescence value of samples)/average fluorescence value of target cells×100%, the killing efficiency was calculated, and the results are shown in FIG. 2.

It can be seen that under various effector-to-target ratios, the CAR-T cells of the present disclosure show a strong killing effect on target cells.

3.2 Detection of Cytokine Release Levels

(1) Collection of Cell Co-Culture Supernatant

Target cells (K562-BCMA cells) or non-target cells (K562 cells, NUGC4 cells) were plated in a 96-well plate at a concentration of 1×10⁵/well, and then NT cells and CAR T cells of the present disclosure were co-cultured with target cells or non-target cells at a ratio of 1:1, and the cell co-culture supernatant was collected after 18-24 hours.

(2) Detection of the Secretion of IL-2 and IFN-γ in the Supernatant by ELISA

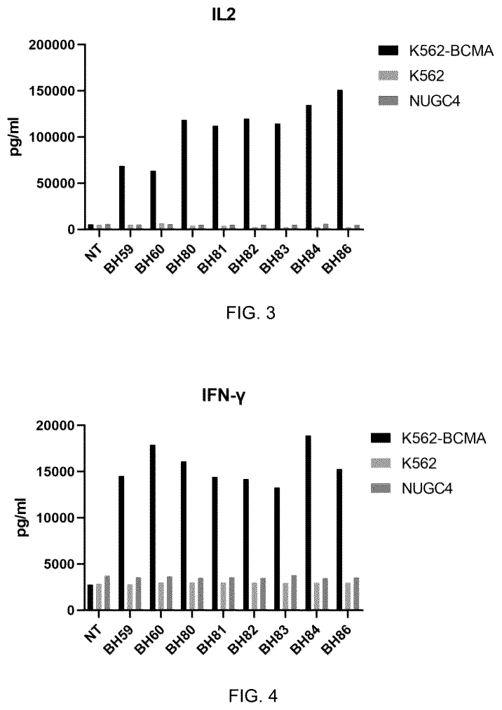

A 96-well plate was coated with Purified anti-human IL2 Antibody (Biolegend, Cat. No. 500302) or Purified anti-human IFN-γ Antibody (Biolegend, Cat. No. 506502) as capture antibody and incubated overnight at 4° C., and then the antibody solution was removed. 250 μL of PBST (1×PBS containing 0.1% Tween) solution containing 2% BSA (sigma, Cat. No. V900933-1 kg) was added, and incubated at 37° C. for 2 hours. The plate was then washed 3 times with 250 μL PBST (1×PBS containing 0.1% Tween). 50 μL of cell co-culture supernatant or standard per well was added and incubated at 37° C. for 1 h, then the plate was washed 3 times with 250 μL of PBST (1×PBS containing 0.1% Tween). Then 50 μL Anti-Interferon gamma antibody [MD-1] (Biotin) (abcam, Cat. No. ab25017) as detection antibody was added to each well, incubated at 37° C. for 1 hour, and the plate was washed 3 times with 250 μL PBST (1×PBS containing 0.1% Tween). Then HRP Streptavidin (Biolegend, Cat. No. 405210) was added, incubated at 37° C. for 30 minutes, and the supernatant was discarded. 250 μL PBST (1×PBS containing 0.1% Tween) was added for washing 5 times. 50 μL of TMB substrate solution was added to each well. Reactions were allowed to occur at room temperature in the dark for 30 minutes, after which 50 μL of 1 mol/L H₂SO₄ was added to each well to stop the reaction. Within 30 minutes of stopping the reaction, a microplate reader was used to detect the absorbance at 450 nm, and the content of cytokines was calculated according to the standard curve (drawn according to the reading value and concentration of the standard), the results are shown in FIG. 3 (IL2) and FIG. 4 (IFN-γ).

As can be seen from FIG. 3 and FIG. 4, compared with NT cells, all CAR-T cells of the present disclosure secreted a large amount of cytokines IL2 and IFN-γ when co-cultured with target cells, and such cytokine release was in a specific manner, as no cytokine release was detectable when co-cultured with non-target cells.

3.3 Detection of Degranulation

The most important way for T cells to kill target cells is cytolytic killing. That is, after T cells come into contact with target cells, they can release a series of cytotoxic granule substances (degranulation), which in turn leads to the lysis of target cells. Lysosome-associated membrane protein 1 (CD107a) is a major component of vesicle membrane proteins. When T cells kill target cells, the toxic granules will reach the cell membrane and fuse with the cell membrane (at this time, the CD107a molecule will be transported to the surface of the cell membrane), causing the release of the granule contents, eventually leading to the death of the target cell. Therefore, the degranulation of T cells can be detected by detecting CD107a, and then the killing effect of T cells can be characterized.

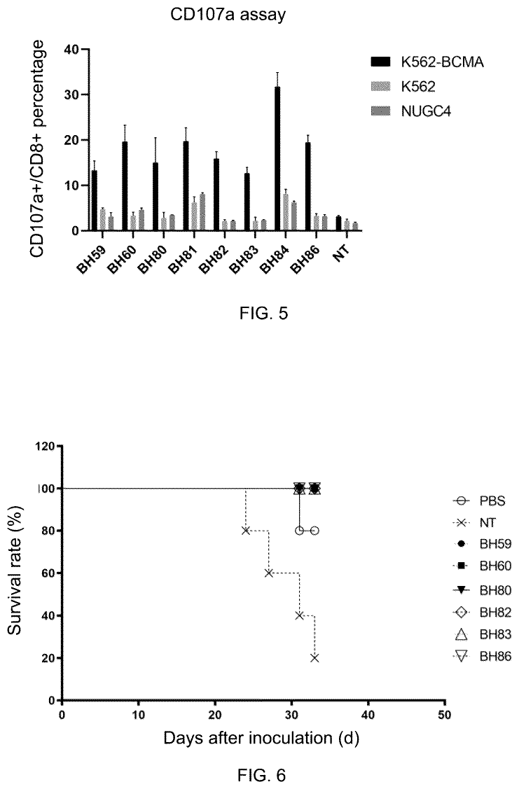

Target cells (K562-BCMA cells) and non-target cells (K562 cells, NUGC4 cells) were plated in a 96-well plate at 1×10⁵/well, respectively, and CAR-T cells and NT cells (negative control) were added at a ratio of 1:1, and then 10 μL PE Mouse anti-human CD107a antibody (BD, Cat. No. 555801) was added to each well, mixed evenly, and incubated at 37° C. and 5% CO₂ in the dark. After 1 hour, 20 μL of Golgi Stop (BD, Cat. No. 51-2092K2) was added to each well, mixed evenly, and incubated at 37° C. and 5% CO₂ in the dark for 2.5 hours. Then, 10 μL of APC anti-human CD8 (BD, Cat. No. 555369) was added to each well, mixed evenly, and incubated at 37° C. and 5% CO₂ in the dark for 0.5 h. The cells were washed twice with 1×PBS, and the cell samples in each well were detected by flow cytometry, and the ratio of CD107a-positive cells to CD8-positive cells was analyzed. The results are shown in FIG. 5.

It can be seen from FIG. 5 that all CAR-T cells of the present disclosure exhibited specific degranulation to target cells.

### Example 4. Tumor Suppression Effect of BCMA CAR-T Cells

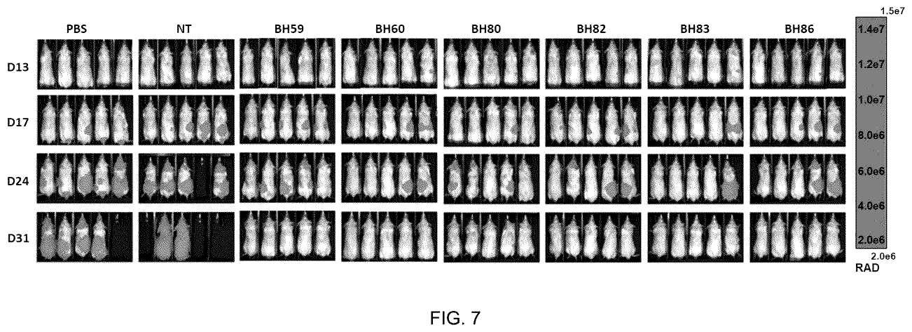

Forty 7-week-old healthy female NCG mice were divided into 8 groups: PBS group, NT group (negative control), BH59 group, BH60 group, BH80 group, BH82 group, BH83 group, BH86 group. On day 0 (D0), 8×10⁶ K562-BCMA cells were injected into the tail vein of each mouse. Fourteen days later (D14), each mouse was injected with PBS solution or 2×10⁶ NT cells or corresponding CAR-T cells into the tail vein according to the grouping situation. Mice were assessed weekly for changes in survival and tumor burden. The statistics of survival percentage data are shown in FIG. 6. Using in vivo optical imaging technology in living animals, the tumor burden expressed in Photons/s in mice was detected on D13, D17, D24, and D31, and the results are shown in FIG. 7.

It can be seen that in the PBS and NT groups, the tumor burden in the mice progressed rapidly and eventually led to the death of the mice. In contrast, after treatment with the CAR-T cells prepared in the present disclosure, the tumor growth of tumor-bearing mice was significantly inhibited, and even the tumor disappeared on D31, so that all mice treated with CAR T cells survived without any death. This shows that the CAR-T cells of the present disclosure can effectively kill tumor target cells, showing a significant treatment effect on tumor.

### Example 5. Humanization of BCMA Single Domain Antibodies

Two single domain antibodies, BH60 and BH86, were humanized as follows: first searching for human antibody sequences with high similarity in the IGBLAST database (https://www.ncbi.nlm nih.gov/igblast/), and then replacing the FR region in the single domain antibody with the corresponding human sequence; then replacing individual amino acid residues according to the different physicochemical properties of the amino acid residues. Finally, 7 BH60 humanized single domain antibodies and 8 BH86 humanized single domain antibodies were obtained, and their amino acid sequences and nucleotide sequences are shown in Table 2 below.

TABLE 2
Humanized anti-BCMA single domain antibody
clones and SEQ ID Nos thereof

| Humanized | | | | | | |
|---|---|---|---|---|---|---|
| single | | | | | | |
| domain | FR (SEQ ID NO) | | | | VHH (SEQ ID NO) | |
| antibody | FR1 | FR2 | FR3 | FR4 | aa | nt |
| BH60_V1 | 43 | 13 | 44 | 45 | 72 | 87 |
| BH60_V2 | 46 | 47 | 48 | 49 | 73 | 88 |
| BH60_V3 | 50 | 47 | 51 | 49 | 74 | 89 |
| BH60_V5 | 52 | 13 | 53 | 45 | 75 | 90 |
| BH60_V6 | 52 | 13 | 54 | 45 | 76 | 91 |
| BH60_GKV1 | 55 | 13 | 51 | 45 | 77 | 92 |
| BH60_GKV2 | 56 | 13 | 51 | 15 | 78 | 93 |
| BH86_V1 | 57 | 9 | 58 | 59 | 79 | 94 |
| BH86_V2 | 60 | 61 | 62 | 63 | 80 | 95 |
| BH86_V4 | 57 | 9 | 58 | 64 | 81 | 96 |
| BH86_V5 | 57 | 9 | 65 | 59 | 82 | 97 |
| BH86_V6 | 57 | 9 | 66 | 64 | 83 | 98 |
| BH86_GKV1 | 67 | 9 | 68 | 24 | 84 | 99 |
| BH86_GKV2 | 67 | 9 | 69 | 70 | 85 | 100 |
| BH86_GKV3 | 67 | 9 | 69 | 71 | 86 | 101 |

### Example 6. Chimeric Antigen Receptor Comprising Humanized Anti-BCMA Single Domain Antibody and its Functional Verification

Sequences encoding the following proteins were synthesized and cloned into pLVX vector (Public Protein/Plasmid Library (PPL), Cat. No.: PPL00157-4a): CD8α signal peptide (SEQ ID NO: 103), humanized anti-BCMA single domain antibody (any sequence selected from SEQ ID NO: 72-86), CD8α hinge region (SEQ ID NO: 105), CD8α transmembrane region (SEQ ID NO: 107), 4-1BB intracellular region (SEQ ID NO: 109) and CD3ζ intracellular region (SEQ ID NO: 111), and the correct insertion of the target sequence was confirmed by sequencing.

Three ml Opti-MEM (Gibco, Cat. No. 31985-070) was added to a sterile tube to dilute the above plasmid, and then packaging vector psPAX2 (Addgene, Cat. No. 12260) and envelope vector pMD2.G (Addgene, Cat. No. 12259) were added according to the ratio of plasmid:viralpackagingvector:viral envelope vector=4:2:1. Then, 120 μl X-treme GENE HP DNA transfection reagent (Roche, Cat. No. 06366236001) was added, mixed immediately, and incubated at room temperature for 15 min, and then the plasmid/vector/transfection reagent mixture was added dropwise to the culture flask of 293T cells. Viruses were collected at 24 hours and 48 hours, pooled, and ultracentrifuged (25000 g, 4° C., 2.5 hours) to obtain concentrated lentiviruses.

T cells were activated with DynaBeads CD3/CD28 CTSTM (Gibco, Cat. No. 40203D) and cultured at 37° C. and 5% CO₂ for 1 day. Then, the concentrated lentivirus was added, and after continuous culture for 3 days, humanized BCMA CAR-T cells containing different humanized BCMA single domain antibodies were obtained. Unmodified wild-type T cells (NT) were used as controls.

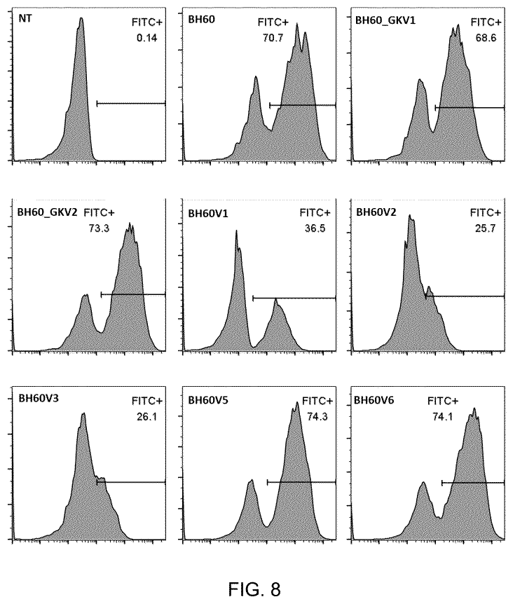

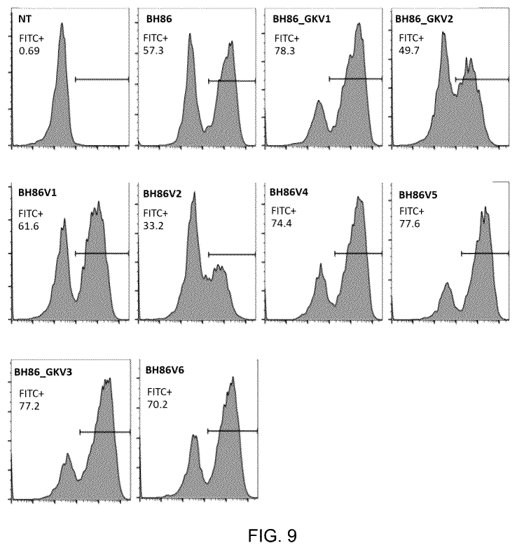

After culturing at 37° C. and 5% CO₂ for 10 days, FITC labeled BCMA (acrobiosystem, Cat. No. BCA-HF254) was used for binding specific staining, and the ability of the corresponding CAR-T cells to specifically bind to the BCMA antigen protein was detected by flow cytometry, which also reflected the expression level of the CAR molecule. The results are shown in FIGS. 8 and 9.

It can be seen that all CAR-T cells tested can successfully express CAR molecules and bind BCMA protein.

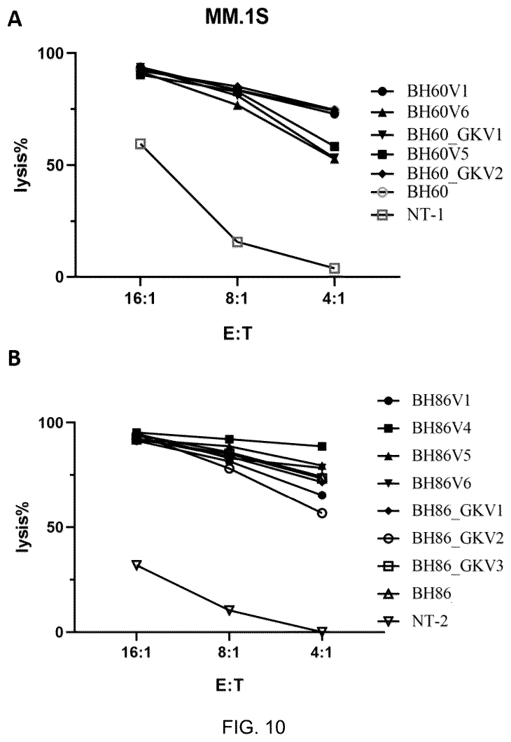

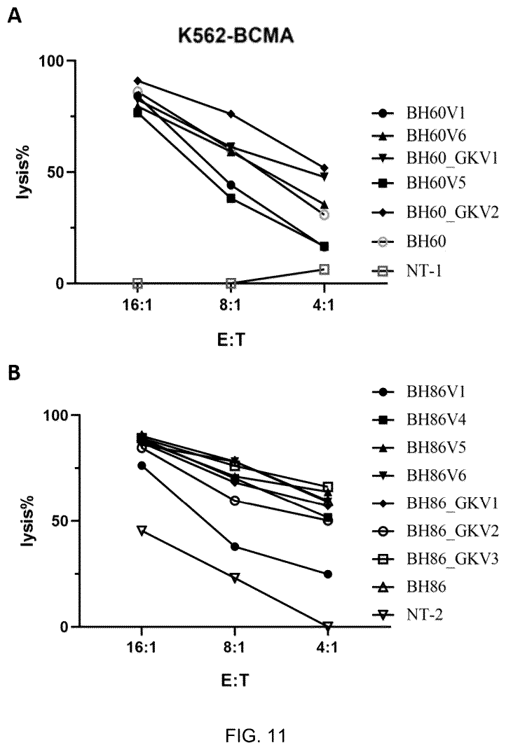

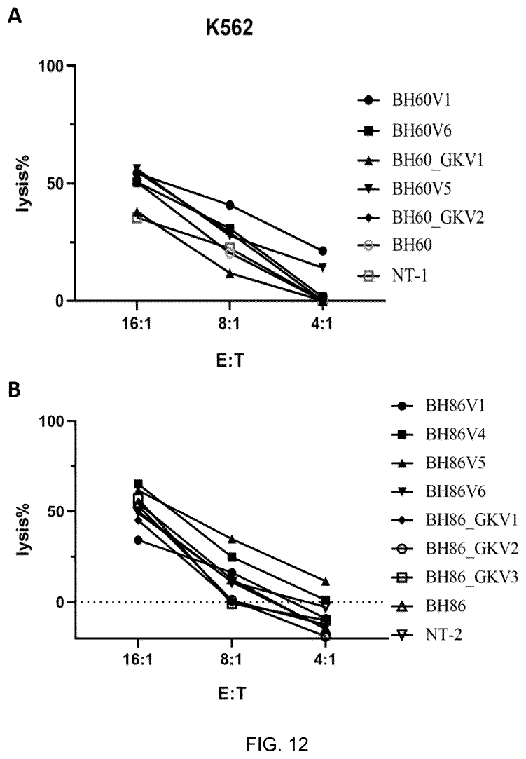

According to the method described in 3.1 of Example 3, the killing effect of CAR-T cells containing humanized anti-BCMA single domain antibodies on target cells (MM.1S and K562-BCMA) was detected, and non-target cell K562 was used as a control. The effector-to-target ratios were 16:1, 8:1, and 4:1, and the results are shown in FIG. 10 (MM.1S), FIG. 11 (K562-BCMA) and FIG. 12 (K562).

It can be seen that under different effector-to-target ratios, CAR-T cells constructed with humanized BCMA single domain antibodies can significantly kill target cells MM.1S and K562-BCMA, but have no obvious killing effect on non-target cells K562 effect, indicating that this killing is specific.

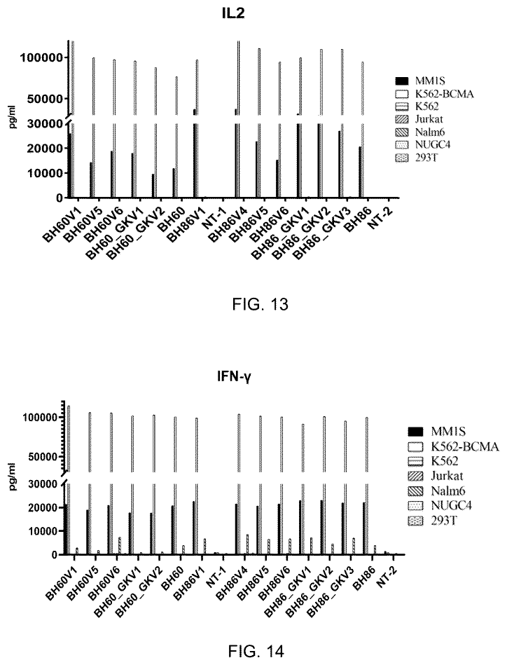

The cytokine release levels after co-culture of CAR-T cells containing humanized anti-BCMA single domain antibody with target cells (MM.1S and K562-BCMA) or non-target cells (K562, Jurkat, Nalm6, NUGC4, 293T) were measured by using human IL-2 DuoSet ELISA Kit (R&D systems, catalog number DY202) or Human IFN-gamma DuoSet ELISA Kit (R&D systems, catalog number DY285), according to the manufacture's recommendations, and the results are shown in FIG. 13 (IL2) and FIG. 14 (IFN-γ).

It can be seen that the CAR-T cells constructed with humanized anti-BCMA single domain antibody released significantly elevated IL2 and IFN-γ only after co-culture with target cells MM.1S and K562-BCMA, while co-culture with all other non-target cells did not result in the release of cytokines.

The inventors also found that CAR-T cells constructed with humanized BCMA single-domain antibodies can kill target cells and release cytokines at a comparable level to CAR-T cells constructed with non-humanized BCMA single-domain antibodies, indicating that humanization has no adverse effect on the killing effect of anti-BCMA single domain antibody in vitro.

### Example 7. Tumor Suppression Effect of Humanized BCMA CAR-T Cells

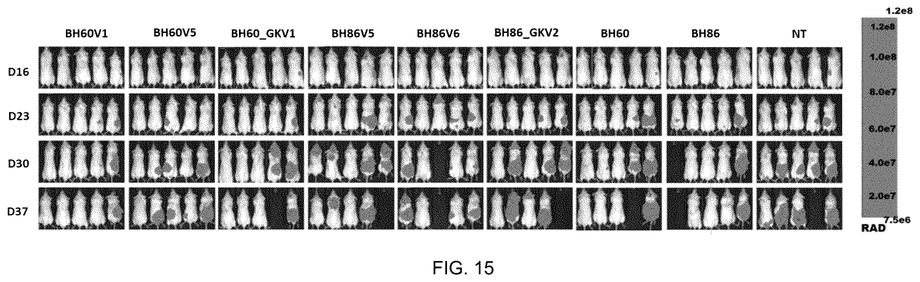

Forty-five 7-week-old healthy female NCG mice were divided into 9 groups: NT group (negative control), BH60V1 group, BH60V5 group, BH60_GKV1 group, BH86V5 group, BH86V6 group, BH86_GKV2 group, BH60 group, BH86 group. On day 0 (DO), 8×10⁶ fluorescein-carrying K562-BCMA cells were injected into the tail vein of each mouse. Sixteen days later (D16), each mouse was injected with PBS solution or 2×10⁶ NT cells or corresponding CAR-T cells into the tail vein according to the grouping situation. Changes in tumor burden in mice were assessed weekly. Using in vivo optical imaging technology in living animals, the tumor burden expressed in Photons/s in mice was detected on D16, D23, D30, and D37. The results are shown in FIG. 15.

It can be seen that the tumor burden in the mice of the NT group continued to progress, and there was no trend of remission. The tumor progression of each group of mice treated with humanized BCMA CAR-T cells was controlled and alleviated to varying degrees, and at least one of the surviving mice showed tumor-free survival. This shows that CAR T cells containing humanized BCMA can effectively inhibit tumor growth, delay tumor progression, and achieve therapeutic effects in vivo.

It should be noted that the above-mentioned are merely for preferred examples of the present disclosure and not used to limit the present disclosure. For one skilled in the art, various modifications and changes may be made to the present disclosure. Those skilled in the art should understand that any amendments, equivalent replacements, improvements, and so on, made within the spirit and principle of the present disclosure, should be covered within the scope of protection of the present disclosure.

1. An anti-BCMA single domain antibody comprising three complementarity determining regions CDR1, CDR2 and CDR3, wherein CDR1 is selected from the group consisting of SEQ ID NO: 1, SEQ ID NO: 4 and SEQ ID NO: 7, CDR2 is selected from the group consisting of SEQ ID NO: 2 and SEQ ID NO: 5, and CDR3 is selected from the group consisting of SEQ ID NO: 3 and SEQ ID NO: 6.

2. The anti-BCMA single domain antibody according to claim 1, wherein the anti-BCMA single domain antibody comprises:
(1) CDR1 as set forth in SEQ ID NO: 1, CDR2 as set forth in SEQ ID NO: 2, and CDR3 as set forth in SEQ ID NO: 3; or
(2) CDR1 as set forth in SEQ ID NO: 4 or SEQ ID NO: 7, CDR2 as set forth in SEQ ID NO: 5, and CDR3 as set forth in SEQ ID NO: 6.

3. The anti-BCMA single domain antibody according to claim 1, wherein the anti-BCMA single domain antibody comprises four framework regions FR1, FR2, FR3 and FR4, wherein FR1 is selected from the group consisting of SEQ ID NO: 8, SEQ ID NO: 12, SEQ ID NO: 17, SEQ ID NO: 20, SEQ ID NO: 22 or variants thereof, FR2 is selected from the group consisting of SEQ ID NO: 9, SEQ ID NO: 13, SEQ ID NO: 18 or variants thereof, FR3 is selected from the group consisting of SEQ ID NO: 10, SEQ ID NO: 14, SEQ ID NO: 19, SEQ ID NO: 21, SEQ ID NO: 23 or variants thereof, and FR4 is selected from the group consisting of SEQ ID NO: 11, SEQ ID NO: 15, SEQ ID NO: 16, SEQ ID NO: 24 or variants thereof, wherein the variant comprises substitution of at most 3 amino acids in the FR.

4. The anti-BCMA single domain antibody according to claim 3, wherein the anti-BCMA single domain antibody comprises:
(1) FR1 as set forth in SEQ ID NO: 8, FR2 as set forth in SEQ ID NO: 9, FR3 as set forth in SEQ ID NO: 10, FR4 as set forth in SEQ ID NO: 11, or variants thereof, wherein the variant comprises substitution of at most 3 amino acids in the FR;
(2) FR1 as set forth in SEQ ID NO: 12 or SEQ ID NO: 22, FR2 as set forth in SEQ ID NO: 13, FR3 as set forth in SEQ ID NO: 14, FR4 as set forth in SEQ ID NO: 15 or SEQ ID NO: 16, or variants thereof, wherein the variant comprises substitution of at most 3 amino acids in the FR;
(3) FR1 as set forth in SEQ ID NO: 17, FR2 as set forth in SEQ ID NO: 18, FR3 as set forth in SEQ ID NO: 19, FR4 as set forth in SEQ ID NO: 15, or variants thereof, wherein the variant comprises substitution of at most 3 amino acids in the FR;
(4) FR1 as set forth in SEQ ID NO: 20, FR2 as set forth in SEQ ID NO: 13, FR3 as set forth in SEQ ID NO: 21, FR4 as set forth in SEQ ID NO: 15 or SEQ ID NO: 16, or variants thereof, wherein the variant comprises substitution of at most 3 amino acids in the FR; or
(5) FR1 as set forth in SEQ ID NO: 8, FR2 as set forth in SEQ ID NO: 9, FR3 as set forth in SEQ ID NO: 23, FR4 as set forth in SEQ ID NO: 11 or SEQ ID NO: 24, or variants thereof, wherein the variant comprises substitution of at most 3 amino acids in the FR.

5. The anti-BCMA single domain antibody according to claim 1, wherein the anti-BCMA single domain antibody has at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NO: 25-33, and specifically binds to BCMA antigen.

6. The anti-BCMA single domain antibody according to claim 1, wherein the anti-BCMA single domain antibody is a natural Camelidae antibody, a camelized antibody, a humanized antibody or a human antibody.

7. The anti-BCMA single domain antibody according to claim 6, wherein the anti-BCMA single domain antibody is a humanized antibody comprising FR1 selected from the group consisting of SEQ ID NO: 43, SEQ ID NO: 46, SEQ ID NO: 50, SEQ ID NO: 52, SEQ ID NO: 55, SEQ ID NO: 56, SEQ ID NO: 57, SEQ ID NO: 60, SEQ ID NO: 67 or variants thereof, FR2 selected from the group consisting of SEQ ID NO: 9, SEQ ID NO: 13, SEQ ID NO: 47, SEQ ID NO: 61 or variants thereof, FR3 selected from the group consisting of SEQ ID NO: 44, SEQ ID NO: 48, SEQ ID NO: 51, SEQ ID NO: 53, SEQ ID NO: 54, SEQ ID NO: 58, SEQ ID NO: 62, SEQ ID NO: 65, SEQ ID NO: 66, SEQ ID NO: 68, SEQ ID NO: 69 or variants thereof, and FR4 selected from the group consisting of SEQ ID NO: 15, SEQ ID NO: 24, SEQ ID NO: 45, SEQ ID NO: 49, SEQ ID NO: 59, SEQ ID NO: 63, SEQ ID NO: 64, SEQ ID NO: 70, SEQ ID NO: 71 or variants thereof, wherein the variant comprises substitution of at most 3 amino acids in the FR.

8. The anti-BCMA single domain antibody according to claim 6, wherein the anti-BCMA single domain antibody is a humanized antibody comprising:
(1) FR1 as set forth in SEQ ID NO: 43, SEQ ID NO: 52, SEQ ID NO: 55 or SEQ ID NO: 56, FR2 as set forth in SEQ ID NO: 13, FR3 as set forth in SEQ ID NO: 44, SEQ ID NO: 51, SEQ ID NO: 53 or SEQ ID NO: 54, FR4 as set forth in SEQ ID NO: 15 or SEQ ID NO: 45, or variants thereof, wherein the variant comprises substitution of at most 3 amino acids in the FR;
(2) FR1 as set forth in SEQ ID NO: 46 or SEQ ID NO: 50, FR2 as set forth in SEQ ID NO: 47, FR3 as set forth in SEQ ID NO: 48 or SEQ ID NO: 51, FR4 as set forth in SEQ ID NO: 49, or variants thereof, wherein the variant comprises substitution of at most 3 amino acids in the FR;
(3) FR1 as set forth in SEQ ID NO: 57 or SEQ ID NO: 67, FR2 as set forth in SEQ ID NO: 9, FR3 as set forth in SEQ ID NO: 58, SEQ ID NO: 65, SEQ ID NO: 66, SEQ ID NO: 68 or SEQ ID NO: 69, FR4 as set forth in SEQ ID NO: 24, SEQ ID NO: 59, SEQ ID NO: 63, SEQ ID NO: 64, SEQ ID NO: 70 or SEQ ID NO: 71, or variants thereof, wherein the variant comprises substitution of at most 3 amino acids in the FR; or
(4) FR1 as set forth in SEQ ID NO: 60, FR2 as set forth in SEQ ID NO: 61, FR3 as set forth in SEQ ID NO: 62, FR4 as set forth in SEQ ID NO: 63, or variants thereof, wherein the variant comprises substitution of at most 3 amino acids in the FR.

9. The anti-BCMA single domain antibody according to claim 6, wherein the anti-BCMA single domain antibody is a humanized antibody having at least 90%, at least 91%, at least 92%, at least 93%, at least 94%, at least 95%, at least 96%, at least 97%, at least 98%, at least 99%, or 100% sequence identity to an amino acid sequence selected from the group consisting of SEQ ID NO: 72-86.

10-11. (canceled)

12. A multispecific antibody, comprising the anti-BCMA single domain antibody according to claim 1, and one or more second antibodies or antigen-binding portions thereof that specifically bind to antigens different from BCMA.

13. The multispecific antibody according to claim 12, wherein the second antibody or antigen-binding portion thereof is selected from the group consisting of a full-length antibody, Fab, Fab′, (Fab′)2, Fv, scFv, scFv-scFv, a minibody, a diabody or sdAb.

14-15. (canceled)

16. A chimeric antigen receptor comprising the anti-BCMA single domain antibody according to claim 1, a transmembrane domain and an intracellular signaling domain.

17. The chimeric antigen receptor according to claim 16, further comprising a co-stimulatory domain selected from the group consisting of CD28 or 4-1BB.

18. (canceled)

19. An engineered immune cell comprising the chimeric antigen receptor according to claim 16.

20. The engineered immune cell according to claim 19, wherein the cell is selected from the group consisting of a T cell, a NK cell, a NKT cell, a macrophage, and a dendritic cell.

21. An antibody conjugate comprising the anti-BCMA single domain antibody according to claim 1 and a second functional structure, wherein the second functional structure is selected from the group consisting of Fc, a radioisotope, a structure moiety for extending half-life, a detectable marker and a drug.

22. The antibody conjugate according to claim 21, wherein the structure moiety for extending half-life is selected from the group consisting of an albumin-binding structure, a transferrin-binding structure, a polyethylene glycol molecule, a recombinant polyethylene glycol molecule, human serum albumin, a fragment of human serum albumin, and a polypeptide binding to human serum albumin; the detectable marker is selected from the group consisting of a fluorophore, a chemiluminescent compound, a bioluminescent compound, an enzyme, an antibiotic resistance gene, and a contrast agent; and the drug is selected from the group consisting of a cytotoxin and an immunomodulator.

23. (canceled)

24. A pharmaceutical composition comprising the anti-BCMA single domain antibody according to claim 1, and one or more pharmaceutically acceptable excipients.

25. A method for treating and/or preventing and/or diagnosing a disease related to BCMA expression, comprising administering to a subject the single domain antibody according to claim 1.

26. The method according to claim 25, wherein the disease associated with BCMA expression is selected from the group consisting of an autoimmune disease, lymphoma, leukemia or plasma cell malignancy.


SEQUENCE LISTING (123 sequences)

SEQ ID NO: 1 - protein, 7 aa, Artificial Sequence
  CDR1
GRAISSI

SEQ ID NO: 2 - protein, 6 aa, Artificial Sequence
  CDR2
NRWGGT

SEQ ID NO: 3 - protein, 10 aa, Artificial Sequence
  CDR3
ASTLDSGYDY

SEQ ID NO: 4 - protein, 7 aa, Artificial Sequence
  CDR1
GGIFTIN

SEQ ID NO: 5 - protein, 5 aa, Artificial Sequence
  CDR2
SRSGS

SEQ ID NO: 6 - protein, 6 aa, Artificial Sequence
  CDR3
DRPLSY

SEQ ID NO: 7 - protein, 7 aa, Artificial Sequence
  CDR1
GITLSSY

SEQ ID NO: 8 - protein, 25 aa, Artificial Sequence
  FR1
QEVLMESGGGLVQAGESLRLSCAAS

SEQ ID NO: 9 - protein, 19 aa, Artificial Sequence
  FR2
VMAWWRQTPGAEREFVASI

SEQ ID NO: 10 - protein, 41 aa, Artificial Sequence
  FR3
PYYSHSVKGRFTITRDHAKNTIHLQMNNLKSADTAVYYCAA

SEQ ID NO: 11 - protein, 10 aa, Artificial Sequence
  FR4
WGQGTLVTVS

SEQ ID NO: 12 - protein, 25 aa, Artificial Sequence
  FR1
QEQLVESGGGSVQPGGSLRLSCVVS

SEQ ID NO: 13 - protein, 19 aa, Artificial Sequence
  FR2
DMAWFRQAPGKQRELVAHI

SEQ ID NO: 14 - protein, 42 aa, Artificial Sequence
  FR3
TYYRDFVKGRFTISRNNAKNTSYLQMNNLKPEDTATYYCNSI

SEQ ID NO: 15 - protein, 9 aa, Artificial Sequence
  FR4
GQGTQVTVS

SEQ ID NO: 16 - protein, 9 aa, Artificial Sequence
  FR4
GQGTLVTVS

SEQ ID NO: 17 - protein, 25 aa, Artificial Sequence
  FR1
QVHLVESGGGLVKAGGSLRLSCVVS

SEQ ID NO: 18 - protein, 19 aa, Artificial Sequence
  FR2
SMAWFRQAPGKQRELVAHI

SEQ ID NO: 19 - protein, 41 aa, Artificial Sequence
  FR3
YYRDFVKGRFTISRNNAKNTSYLQMNNLKPEDTATYYCNSI

SEQ ID NO: 20 - protein, 25 aa, Artificial Sequence
  FR1
QERLVESGGGSVHAGGSLRLSCVAS

SEQ ID NO: 21 - protein, 42 aa, Artificial Sequence
  FR3
TYYADFVKGRFTISKNNAKNTSYLQMNSLKPDDTATYYCNSI

SEQ ID NO: 22 - protein, 25 aa, Artificial Sequence
  FR1
QVQLVESGGGSVQPGGSLRLSCVVS

SEQ ID NO: 23 - protein, 41 aa, Artificial Sequence
  FR3
PYYSNSVKGRFAITRDHAKNTIHLQMNNLKSADTAVYYCAA

SEQ ID NO: 24 - protein, 10 aa, Artificial Sequence
  FR4
WGHGTQVTVS

SEQ ID NO: 25 - protein, 118 aa, Artificial Sequence
  BH59
QEVLMESGGGLVQAGESLRLSCAASGRAISSIVMAWWRQTPGAEREFVASINRWGGTPYYSHSVKGRFTITRDHAKNTIHLQMNNLKSADTAVYYCAAASTLDSGYDYWGQGTLVTVS

SEQ ID NO: 26 - protein, 113 aa, Artificial Sequence
  BH60
QEQLVESGGGSVQPGGSLRLSCVVSGGIFTINDMAWFRQAPGKQRELVAHISRSGSTYYRDFVKGRFTISRNNAKNTSYLQMNNLKPEDTATYYCNSIDRPLSYGQGTQVTVS

SEQ ID NO: 27 - protein, 113 aa, Artificial Sequence
  BH80
QEQLVESGGGSVQPGGSLRLSCVVSGGIFTINDMAWFRQAPGKQRELVAHISRSGSTYYRDFVKGRFTISRNNAKNTSYLQMNNLKPEDTATYYCNSIDRPLSYGQGTLVTVS

SEQ ID NO: 28 - protein, 113 aa, Artificial Sequence
  BH81
QVHLVESGGGLVKAGGSLRLSCVVSGITLSSYSMAWFRQAPGKQRELVAHISRSGSTYYRDFVKGRFTISRNNAKNTSYLQMNNLKPEDTATYYCNSIDRPLSYGQGTQVTVS

SEQ ID NO: 29 - protein, 113 aa, Artificial Sequence
  BH82
QERLVESGGGSVHAGGSLRLSCVASGGIFTINDMAWFRQAPGKQRELVAHISRSGSTYYADFVKGRFTISKNNAKNTSYLQMNSLKPDDTATYYCNSIDRPLSYGQGTLVTVS

SEQ ID NO: 30 - protein, 113 aa, Artificial Sequence
  BH83
QERLVESGGGSVHAGGSLRLSCVASGGIFTINDMAWFRQAPGKQRELVAHISRSGSTYYADFVKGRFTISKNNAKNTSYLQMNSLKPDDTATYYCNSIDRPLSYGQGTQVTVS

SEQ ID NO: 31 - protein, 113 aa, Artificial Sequence
  BH84
QVQLVESGGGSVQPGGSLRLSCVVSGGIFTINDMAWFRQAPGKQRELVAHISRSGSTYYRDFVKGRFTISRNNAKNTSYLQMNNLKPEDTATYYCNSIDRPLSYGQGTQVTVS

SEQ ID NO: 32 - protein, 118 aa, Artificial Sequence
  BH86
QEVLMESGGGLVQAGESLRLSCAASGRAISSIVMAWWRQTPGAEREFVASINRWGGTPYYSNSVKGRFAITRDHAKNTIHLQMNNLKSADTAVYYCAAASTLDSGYDYWGHGTQVTVS

SEQ ID NO: 33 - protein, 118 aa, Artificial Sequence
  BH87
QEVLMESGGGLVQAGESLRLSCAASGRAISSIVMAWWRQTPGAEREFVASINRWGGTPYYSNSVKGRFAITRDHAKNTIHLQMNNLKSADTAVYYCAAASTLDSGYDYWGQGTLVTVS

SEQ ID NO: 34 - DNA, 354 bp, Artificial Sequence
  BH59
caggaggtactgatggagtctgggggaggattggtgcaggccggggagtctctaagactatcctgtgcagcctctggacgcgccattagtagtattgtcatggcctggtggcgccagactccaggggcggaacgcgagtttgtcgcctccatcaatcggtggggtggtacgccatactattcacactccgtgaagggccgattcaccatcaccagggatcatgccaagaatacgattcatctgcaaatgaacaatctgaaatctgcggacacggccgtttactactgcgctgcagcgtcgacgttggattcggggtatgactactggggccaggggacccttgtcaccgtctcc

SEQ ID NO: 35 - DNA, 339 bp, Artificial Sequence
  BH60
caggagcagctggtggagtctgggggaggctcggtgcagcctggggggtctctgagactctcctgtgtagtctctggaggcatcttcacgatcaatgacatggcctggttccgccaggctccagggaaacagcgcgagttggtcgcccatattagtagaagtggtagcacatactatagagacttcgtgaagggccgattcaccatctccagaaacaatgccaagaacacgtcatatctgcaaatgaataacctgaaacctgaggacacggccacatattactgtaattcgatcgaccggcccttatcttacggccaggggacccaggtcaccgtctcc

SEQ ID NO: 36 - DNA, 339 bp, Artificial Sequence
  BH80
caggagcagctggtggagtctgggggaggctcggtgcagcctggggggtctctgagactctcctgtgtagtctctggaggcatcttcacgatcaatgacatggcctggttccgccaggctccagggaaacagcgcgagttggtcgcccatattagtagaagtggtagcacatactatagagacttcgtgaagggccgattcaccatctccagaaacaatgccaagaacacgtcatatctgcaaatgaataacctgaaacctgaggacacggccacatattactgtaattcgatcgaccggcccttatcttacggccaggggaccctagtcactgtctcc

SEQ ID NO: 37 - DNA, 339 bp, Artificial Sequence
  BH81
caggtgcacctggtagagtctgggggaggattggtcaaggctgggggctctctgagactctcctgtgtagtctctggaattaccttgagtagttattccatggcctggttccgccaggctccagggaaacagcgcgagttggtcgcccatattagtagaagtggtagcacatactatagagacttcgtgaagggccgattcaccatctccagaaacaatgccaagaacacgtcatatctgcaaatgaataacctgaaacctgaggacacggccacatattactgtaattcgatcgaccggcccttatcttacggccaggggacccaggtcaccgtctcc

SEQ ID NO: 38 - DNA, 339 bp, Artificial Sequence
  BH82
caggagcgcctggtggagtctgggggaggctcggtgcacgctggggggtctctgagactctcctgtgtagcctctggaggcatcttcacgatcaatgacatggcctggttccgccaggctccagggaagcagcgcgagttggtcgcccatatttccagaagtggtagcacatactatgcagacttcgtgaagggccgattcaccatctccaaaaacaatgccaagaacacgtcatatctgcaaatgaacagcctgaaacctgacgacacggccacatattattgtaattcgatcgaccggcccttatcttatggccaggggaccctggtcaccgtctcc

SEQ ID NO: 39 - DNA, 339 bp, Artificial Sequence
  BH83
caggagcgcctggtggagtctgggggaggctcggtgcacgctggggggtctctgagactctcctgtgtagcctctggaggcatcttcacgatcaatgacatggcctggttccgccaggctccagggaagcagcgcgagttggtcgcccatatttccagaagtggtagcacatactatgcagacttcgtgaagggccgattcaccatctccaaaaacaatgccaagaacacgtcatatctgcaaatgaacagcctgaaacctgacgacacggccacatattattgtaattcgatcgaccggcccttatcttatggccaggggacccaggtcaccgtctcc

SEQ ID NO: 40 - DNA, 339 bp, Artificial Sequence
  BH84
caggtgcagctggtggagtctgggggaggctcggtgcagcctggggggtctctgagactctcctgtgtagtctctggaggcatcttcacgatcaatgacatggcctggttccgccaggctccagggaaacagcgcgagttggtcgcccatattagtagaagtggtagcacatactatagagacttcgtgaagggccgattcaccatctccagaaacaatgccaagaacacgtcatatctgcaaatgaataacctgaaacctgaggacacggccacatattactgtaattcgatcgaccggcccttatcttacggccaggggacccaggtcaccgtctcc

SEQ ID NO: 41 - DNA, 354 bp, Artificial Sequence
  BH86
caggaggtactgatggagtctgggggaggattggtgcaggccggggagtctctaagactatcctgtgcagcctctggacgcgccattagtagtattgtcatggcctggtggcgccagactccaggggcggaacgcgagtttgtcgcctccatcaatcggtggggtggtacgccatactattcgaactccgtgaagggccgattcgccatcaccagggatcatgccaagaatacgattcatctgcaaatgaacaatctgaaatctgcggacacggccgtttactactgcgctgcagcgtcgacgttggattcggggtatgactactggggccacgggacccaagtcaccgtctcc

SEQ ID NO: 42 - DNA, 354 bp, Artificial Sequence
  BH87
caggaagtgctgatggaaagcggcggcggcctggtgcaggcgggcgaaagcctgcgcctgagctgcgcggcgagcggccgcgcgattagcagcattgtgatggcgtggtggcgccagaccccgggcgcggaacgcgaatttgtggcgagcattaaccgctggggcggcaccccgtattatagcaacagcgtgaaaggccgctttgcgattacccgcgatcatgcgaaaaacaccattcatctgcagatgaacaacctgaaaagcgcggataccgcggtgtattattgcgcggcggcgagcaccctggatagcggctatgattattggggccagggcaccctggtgaccgtgagc

SEQ ID NO: 43 - protein, 25 aa, Artificial Sequence
  Humanized FR1
QVQLVESGGGLVQPGGSLRLSCAVS

SEQ ID NO: 44 - protein, 42 aa, Artificial Sequence
  Humanized FR3
TYYRDSVKGRFTISRDNSKNTLYLQMNSLRAEDTATYYCNSI

SEQ ID NO: 45 - protein, 9 aa, Artificial Sequence
  BH60 Humanized FR4
GQGTLVTVS

SEQ ID NO: 46 - protein, 25 aa, Artificial Sequence
  Humanized FR1
QVQLVESGGGVVQPGGSLRLSCVVS

SEQ ID NO: 47 - protein, 19 aa, Artificial Sequence
  Humanized FR2
DMAWVRQAPGKGLEWVAHI

SEQ ID NO: 48 - protein, 42 aa, Artificial Sequence
  Humanized FR3
TYYRDSVKGRFTISRDNSKNTLYLQMNSLRAEDTAVYYCNSI

SEQ ID NO: 49 - protein, 10 aa, Artificial Sequence
  Humanized FR4
GQGTLVTVSS

SEQ ID NO: 50 - protein, 25 aa, Artificial Sequence
  Humanized FR1
EVQLVESGGGLVQPGGSLRLSCVVS

SEQ ID NO: 51 - protein, 42 aa, Artificial Sequence
  Humanized FR3
TYYRDSVKGRFTISRDNAKNSLYLQMNSLRAEDTAVYYCNSI

SEQ ID NO: 52 - protein, 25 aa, Artificial Sequence
  Humanized FR1
QVQLVESGGGLVQPGGSLRLSCVVS

SEQ ID NO: 53 - protein, 42 aa, Artificial Sequence
  Humanized FR3
TYYRDFVKGRFTISRDNSKNTLYLQMNSLRAEDTATYYCNSI

SEQ ID NO: 54 - protein, 42 aa, Artificial Sequence; identical to SEQ ID NO: 53
  Humanized FR3

SEQ ID NO: 55 - protein, 25 aa, Artificial Sequence
  Humanized FR1
QVQLVESGGGLVQPGGSLRLSCVAS

SEQ ID NO: 56 - protein, 25 aa, Artificial Sequence
  Humanized FR1
QVQLVESGGGLVQPGGSLRLSCAAS

SEQ ID NO: 57 - protein, 25 aa, Artificial Sequence
  Humanized FR1
QVQLVESGGGLVQAGESLRLSCAAS

SEQ ID NO: 58 - protein, 41 aa, Artificial Sequence
  Humanized FR3
PYYSDSVKGRFTISRDNSKNTLYLQMNSLRAEDTAVYYCAA

SEQ ID NO: 59 - protein, 10 aa, Artificial Sequence
  Humanized FR4
WGQGTLVTVS

SEQ ID NO: 60 - protein, 25 aa, Artificial Sequence
  Humanized FR1
EVQLVESGGGLVKPGGSLRLSCAAS

SEQ ID NO: 61 - protein, 19 aa, Artificial Sequence
  Humanized FR2
VMAWVRQAPGKGLEWVASI

SEQ ID NO: 62 - protein, 41 aa, Artificial Sequence
  Humanized FR3
PYYSAPVKGRFTISRDDSKNTLYLQMNSLKTEDTAVYYCAA

SEQ ID NO: 63 - protein, 11 aa, Artificial Sequence
  Humanized FR4
WGQGTLVTVSS

SEQ ID NO: 64 - protein, 9 aa, Artificial Sequence
  Humanized FR4
GHGTLVTVS

SEQ ID NO: 65 - protein, 41 aa, Artificial Sequence
  Humanized FR3
PYYSNSVKGRFTISRDNSKNTIHLQMNSLRSEDTAVYYCAA

SEQ ID NO: 66 - protein, 41 aa, Artificial Sequence
  Humanized FR3
PYYSNSVKGRFAISRDNSKNTIHLQMNSLRSEDTAVYYCAA

SEQ ID NO: 67 - protein, 25 aa, Artificial Sequence
  Humanized FR1
QEVLVESGGGLVQPGGSLRLSCAAS

SEQ ID NO: 68 - protein, 41 aa, Artificial Sequence
  Humanized FR3
PYYSDSVKGRFTISRDHAKNTLYLQMNSLRAEDTAVYYCAA

SEQ ID NO: 69 - protein, 41 aa, Artificial Sequence
  Humanized FR3
PYYSDSVKGRFTISRDHAKNTLYLQMNSLKSADTAVYYCAA

SEQ ID NO: 70 - protein, 9 aa, Artificial Sequence
  Humanized FR4
GPGTQVTVS

SEQ ID NO: 71 - protein, 9 aa, Artificial Sequence
  Humanized FR4
GQGTQVTVS

SEQ ID NO: 72 - protein, 113 aa, Artificial Sequence
  BH60V1
QVQLVESGGGLVQPGGSLRLSCAVSGGIFTINDMAWFRQAPGKQRELVAHISRSGSTYYRDSVKGRFTISRDNSKNTLYLQMNSLRAEDTATYYCNSIDRPLSYGQGTLVTVS

SEQ ID NO: 73 - protein, 113 aa, Artificial Sequence; identical to SEQ ID NO: 72
  BH60V2

SEQ ID NO: 74 - protein, 114 aa, Artificial Sequence
  BH60V3
EVQLVESGGGLVQPGGSLRLSCVVSGGIFTINDMAWVRQAPGKGLEWVAHISRSGSTYYRDSVKGRFTISRDNAKNSLYLQMNSLRAEDTAVYYCNSIDRPLSYGQGTLVTVSS

SEQ ID NO: 75 - protein, 113 aa, Artificial Sequence
  BH60V5
QVQLVESGGGLVQPGGSLRLSCVVSGGIFTINDMAWFRQAPGKQRELVAHISRSGSTYYRDFVKGRFTISRDNSKNTLYLQMNSLRAEDTATYYCNSIDRPLSYGQGTLVTVS

SEQ ID NO: 76 - protein, 113 aa, Artificial Sequence
  BH60V6
QVQLVESGGGLVQPGGSLRLSCVVSGGIFTINDMAWFRQAPGKQRELVAHISRSGSTYYRDFVKGRFTISRDNSKNTSYLQMNNLRAEDTATYYCNSIDRPLSYGQGTLVTVS

SEQ ID NO: 77 - protein, 113 aa, Artificial Sequence
  BH60_GKV1
QVQLVESGGGLVQPGGSLRLSCVASGGIFTINDMAWFRQAPGKQRELVAHISRSGSTYYRDSVKGRFTISRDNAKNSLYLQMNSLRAEDTAVYYCNSIDRPLSYGQGTLVTVS

SEQ ID NO: 78 - protein, 113 aa, Artificial Sequence
  BH60_GKV2
QVQLVESGGGLVQPGGSLRLSCAASGGIFTINDMAWFRQAPGKQRELVAHISRSGSTYYRDSVKGRFTISRDNAKNSLYLQMNSLRAEDTAVYYCNSIDRPLSYGQGTQVTVS

SEQ ID NO: 79 - protein, 118 aa, Artificial Sequence
  BH86V1
QVQLVESGGGLVQAGESLRLSCAASGRAISSIVMAWWRQTPGAEREFVASINRWGGTPYYSDSVKGRFTISRDNSKNTLYLQMNSLRAEDTAVYYCAAASTLDSGYDYWGQGTLVTVS

SEQ ID NO: 80 - protein, 119 aa, Artificial Sequence
  BH86V2
EVQLVESGGGLVKPGGSLRLSCAASGRAISSIVMAWVRQAPGKGLEWVASINRWGGTPYYSAPVKGRFTISRDDSKNTLYLQMNSLKTEDTAVYYCAAASTLDSGYDYWGQGTLVTVSS

SEQ ID NO: 81 - protein, 118 aa, Artificial Sequence
  BH86V4
QVQLVESGGGLVQAGESLRLSCAASGRAISSIVMAWWRQTPGAEREFVASINRWGGTPYYSDSVKGRFTISRDNSKNTLYLQMNSLRAEDTAVYYCAAASTLDSGYDYWGHGTLVTVS

SEQ ID NO: 82 - protein, 118 aa, Artificial Sequence; identical to SEQ ID NO: 81
  BH86V5

SEQ ID NO: 83 - protein, 118 aa, Artificial Sequence
  BH86V6
QVQLVESGGGLVQAGESLRLSCAASGRAISSIVMAWWRQTPGAEREFVASINRWGGTPYYSNSVKGRFAISRDNSKNTIHLQMNSLRSEDTAVYYCAAASTLDSGYDYWGHGTLVTVS

SEQ ID NO: 84 - protein, 118 aa, Artificial Sequence
  BH86_GKV1
QEVLVESGGGLVQPGGSLRLSCAASGRAISSIVMAWWRQTPGAEREFVASINRWGGTPYYSDSVKGRFTISRDHAKNTLYLQMNSLRAEDTAVYYCAAASTLDSGYDYWGHGTQVTVS

SEQ ID NO: 85 - protein, 118 aa, Artificial Sequence
  BH86_GKV2
QEVLVESGGGLVQPGGSLRLSCAASGRAISSIVMAWWRQTPGAEREFVASINRWGGTPYYSDSVKGRFTISRDHAKNTLYLQMNSLKSADTAVYYCAAASTLDSGYDYWGPGTQVTVS

SEQ ID NO: 86 - protein, 118 aa, Artificial Sequence
  BH86_GKV3
QEVLVESGGGLVQPGGSLRLSCAASGRAISSIVMAWWRQTPGAEREFVASINRWGGTPYYSDSVKGRFTISRDHAKNTLYLQMNSLKSADTAVYYCAAASTLDSGYDYWGQGTQVTVS

SEQ ID NO: 87 - DNA, 339 bp, Artificial Sequence
  BH60V1
caggtgcagctggtagagagcggaggtggtctcgtacaaccgggaggatctctcaggctctcatgtgcggtcagtggaggcatctttacaattaacgacatggcctggtttaggcaagctccgggaaaacaaagagagcttgttgcgcatatttctcggtcagggagtacatactaccgggacagcgttaaaggaaggtttactatatctcgggacaatagtaagaacactctgtacttgcagatgaactcccttagagcagaggataccgcaacttattactgcaatagtattgaccgccctctcagttacggccagggtactctggtgacagtgagc

SEQ ID NO: 88 - DNA, 342 bp, Artificial Sequence
  BH60V2
caggtgcaacttgtcgaaagtgggggaggagtagttcaacccggcgggtcactgagactgtcttgcgtcgtcagcggcggtatttttaccataaacgatatggcctgggtccggcaggcgccaggtaaaggcctggaatgggtcgcgcacatctcccgaagtggctccacgtactatagggattctgtaaaggggcgctttacaatatcaagggacaatagcaaaaatacactctacctgcaaatgaactctctgcgggctgaagacacggcagtatattattgcaatagcattgaccggcccctgtcatacggccaaggaacgctcgttacggtctccagc

SEQ ID NO: 89 - DNA, 342 bp, Artificial Sequence; identical to SEQ ID NO: 88
  BH60V3

SEQ ID NO: 90 - DNA, 339 bp, Artificial Sequence
  BH60V5
caggtccagttggttgagtcaggagggggattggtacagccaggcggaagtttgcgcctttcatgtgtggtttccgggggcatcttcactataaacgatatggcctggttccgccaagctcctgggaaacaacgcgaacttgtagcccatatatcccgatccggcagtacatattaccgagacttcgtcaaagggcgctttacaattagccgagacaattctaaaaacacgctctatttgcaaatgaactctttgcgggccgaagatacagcgacttactactgcaactccatcgacagaccgctctcttacgggcaagggactcttgtgacggtaagc

SEQ ID NO: 91 - DNA, 339 bp, Artificial Sequence
  BH60V6
caggtgcagctggtggaaagcggcggcggcctggtgcagccgggcggcagcctgcgcctgagctgcgtggtgagcggcggcatttttaccattaacgatatggcgtggtttcgccaggcgccgggcaaacagcgcgaactggtggcgcatattagccgcagcggcagcacctattatcgcgattttgtgaaaggccgctttaccattagccgcgataacagcaaaaacaccagctatctgcagatgaacaacctgcgcgcggaagataccgcgacctattattgcaacagcattgatcgcccgctgagctatggccagggcaccctggtgaccgtgagc

SEQ ID NO: 92 - DNA, 339 bp, Artificial Sequence; identical to SEQ ID NO: 91
  BH60_GKV1

SEQ ID NO: 93 - DNA, 339 bp, Artificial Sequence
  BH60_GKV2
caggttcaactggtagaaagcggcggcggcttggttcaacctggcggttctctgaggctttcttgtgcagcatctggtgggatattcacgattaatgatatggcctggtttaggcaagcgccaggaaaacagcgggagcttgtggcacatatatctaggagcgggagtacctactatagagacagcgtgaaggggagattcactatttctagggacaacgctaagaacagtctctacctgcaaatgaacagccttagagccgaggacaccgctgtctattattgtaatagcatagataggcccctgagttacgggcaagggacgcaagtcacggtaagc

SEQ ID NO: 94 - DNA, 354 bp, Artificial Sequence
  BH86V1
caggtgcagcttgtggaaagcggcggagggttggtccaagccggggagtctcttagactgagttgtgctgcaagcggacgggcaatatcaagtatcgtcatggcttggtggaggcagacacctggcgctgagagggagttcgtcgcctctataaacaggtggggtgggactccgtattattccgattctgtaaaaggaagattcactatttccagggataattcaaaaaacaccctctatcttcagatgaactcacttcgagcagaagatacagctgtctactactgtgcggctgcaagtacactggattcaggatatgactattggggccagggaactctggtaaccgtgagc

SEQ ID NO: 95 - DNA, 357 bp, Artificial Sequence
  BH86V2
gaagtgcaacttgttgagagcggaggtggtcttgtaaaacccggcgggtctttgcgactctcctgtgccgcaagtggccgggccatcagctccattgtaatggcgtgggtaagacaagcgcctggaaaggggttggagtgggtagcttcaataaaccggtggggcggcacgccctactatagtgcgcccgtaaaaggacgattcaccatatctcgggatgacagtaagaatactctgtatctccaaatgaatagccttaaaacggaagatacagcggtatactattgtgctgctgcgagcacattggattcaggctacgactattggggccagggcaccctcgtgacagtgtctagc

SEQ ID NO: 96 - DNA, 354 bp, Artificial Sequence
  BH86V4
caggtccagcttgttgagtctggcggaggtctggtacaggccggggagtcactgaggcttagctgtgcagcatccggtcgggccatatccagtatcgttatggcttggtggcgccaaacgcctggtgcggaacgggagttcgtagcaagcatcaaccgctggggaggaactccatattattctgacagcgtgaagggccggttcacgataagtagagataactctaagaacaccctgtacctccaaatgaatagcctccgggcagaggatacggcagtgtattactgtgcggcggcatctacgctggactcaggctacgattactggggccacgggactttggtcacggtgagc

SEQ ID NO: 97 - DNA, 354 bp, Artificial Sequence
  BH86V5
caggtgcaactcgtggaatcaggaggcggactggtgcaagccggagaatcactccgcctcagttgtgccgcatcaggtcgagccataagttcaattgtgatggcgtggtggaggcagacccctggtgccgaacgcgaatttgtggcctctattaacagatggggtggaacgccctattactcaaactctgtgaaaggtcgatttaccattagccgggacaacagtaagaatactatacacctgcaaatgaactccctcaggagcgaagatacggcagtttattattgtgctgcggcgagcacgcttgactctggatacgactattggggtcaaggaacactcgtgacggtcagc

SEQ ID NO: 98 - DNA, 354 bp, Artificial Sequence
  BH86V6
caggtacagctggttgagtccggtgggggtctcgtccaggctggcgagtccttgagactttcttgcgccgcgtctggacgagctatttctagtatcgttatggcgtggtggagacagactcctggagcagaacgcgagtttgtcgcaagcatcaatagatggggagggacgccctactactctaattctgtaaaaggccgctttgcaatttctagggataattctaagaatacaatccatcttcagatgaactccctgagatcagaggacacggctgtttattactgtgcggcagcgtcaacactggattccgggtatgactattggggacacgggacgcttgtcaccgtcagc

SEQ ID NO: 99 - DNA, 354 bp, Artificial Sequence; identical to SEQ ID NO: 98
  BH86_GKV1

SEQ ID NO: 100 - DNA, 354 bp, Artificial Sequence
  BH86_GKV2
caggaggtgcttgtagaaagtggaggggggcttgtacaacctggaggtagccttaggttgtcttgcgctgcttcaggcagagctatcagcagcatagtgatggcttggtggagacaaacccccggcgccgagcgggaatttgttgcgtcaataaatcgctggggtggcactccgtactattcagactccgttaaaggccggtttaccattagtagagaccacgcaaaaaataccctttatttgcaaatgaactctctcaagagcgccgacacagctgtatattactgtgcagcagcttccacccttgactctggttatgactattggggacctgggacgcaggtgacagttagc

SEQ ID NO: 101 - DNA, 354 bp, Artificial Sequence
  BH86_GKV3
caggaagttctcgtcgaaagcgggggtggtctcgttcaacctggaggctctctgcggctcagttgtgcagcgtctggtcgagccatcagttccatagtaatggcttggtggcgccaaacacctggagctgagagagagttcgtagccagtataaatcgatggggcggaaccccatattactcagactctgtaaaagggcgattcaccatcagccgagaccacgcgaaaaacactctttacttgcaaatgaacagcttgaaatcagctgatacggctgtttactattgcgcggctgcttctacactggatagtggttatgactattggggacaggggactcaagtaactgtcagc

SEQ ID NO: 102 - DNA, 63 bp, Artificial Sequence
  CD8alpha signal peptide
atggccttaccagtgaccgccttgctcctgccgctggccttgctgctccacgccgccaggccg

SEQ ID NO: 103 - protein, 21 aa, Artificial Sequence
  CD8alpha signal peptide
MALPVTALLLPLALLLHAARP

SEQ ID NO: 104 - DNA, 135 bp, Artificial Sequence
  CD8alpha hinge region
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 105 - protein, 45 aa, Artificial Sequence
  CD8alpha hinge region
TTTPAPRPPTPAPTIASQPLSLRPEACRPAAGGAVHTRGLDFACD

SEQ ID NO: 106 - DNA, 75 bp, Artificial Sequence
  CD8alpha transmembrane domain
atctacatctgggcgcccttggccgggacttgtggggtccttctcctgtcactggttatcaccctttactgcaaa

SEQ ID NO: 107 - protein, 25 aa, Artificial Sequence
  CD8alpha transmembrane domain
IYIWAPLAGTCGVLLLSLVITLYCK

SEQ ID NO: 108 - DNA, 120 bp, Artificial Sequence
  4-1BB costimulatory domain
cggggcagaaagaaactcctgtatatattcaaacaaccatttatgagaccagtacaaactactcaagaggaagatggctgtagctgccgatttccagaagaagaagaaggaggatgtgaa

SEQ ID NO: 109 - protein, 40 aa, Artificial Sequence
  4-1BB costimulatory domain
RGRKKLLYIFKQPFMRPVQTTQEEDGCSCRFPEEEEGGCE

SEQ ID NO: 110 - DNA, 339 bp, Artificial Sequence
  CD3zeta intracellular signaling domain
ctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 111 - protein, 113 aa, Artificial Sequence
  CD3zeta intracellular signaling domain
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - DNA, 123 bp, Artificial Sequence
  CD28 costimulatory domain
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 113 - protein, 41 aa, Artificial Sequence
  CD28 costimulatory domain
RSKRSRLLHSDYMNMTPRRPGPTRKHYQPYAPPRDFAAYRS

SEQ ID NO: 114 - protein, 27 aa, Artificial Sequence
  CD28 transmembrane domain
FWVLVVVGGVLACYSLLVTVAFIIFWV

SEQ ID NO: 115 - DNA, 81 bp, Artificial Sequence
  CD28 transmembrane domain
ttttgggtcctcgtcgtagttggaggggtacttgcctgttatagcctcctggttaccgtagcatttattatattctgggtg

SEQ ID NO: 116 - protein, 114 aa, Artificial Sequence
  CD3zeta intracellular signaling domain
LRVKFSRSADAPAYQQGQNQLFNELNLGRREEFDVLDKRRGRDPEMGGKPQRRKNPQEGLYNELQKDKMAEAYSEIGMKGERRRGKGHDGLFQGLSTATKDTFDALHMQALPPR

SEQ ID NO: 117 - DNA, 342 bp, Artificial Sequence
  CD3zeta intracellular signaling domain
ctgagagtgaagttcagcaggagcgcagacgcccccgcgtaccagcagggccagaaccagctctttaacgagctcaatctaggacgaagagaggagttcgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggccttttccagggtctcagtacagccaccaaggacacctttgacgcccttcacatgcaggccctgccccctcgc

SEQ ID NO: 118 - protein, 20 aa, Artificial Sequence
  B2M signal peptide
MSRSVALAVLALLSLSGLEA

SEQ ID NO: 119 - DNA, 60 bp, Artificial Sequence
  B2M signal peptide
atgtcccgctctgttgctttggctgtgctggcccttttgtcccttagcggactggaggcc

SEQ ID NO: 120 - protein, 39 aa, Artificial Sequence
  CD28 hinge region
IEVMYPPPYLDNEKSNGTIIHVKGKHLCPSPLFPGPSKP

SEQ ID NO: 121 - DNA, 117 bp, Artificial Sequence
  CD28 hinge region
attgaagttatgtatcctcctccttacctagacaatgagaagagcaatggaaccattatccatgtgaaagggaaacacctttgtccaagtcccctatttcccggaccttctaagccc

SEQ ID NO: 122 - protein, 12 aa, Artificial Sequence
  IgG4 hinge region
ESKYGPPCPPCP

SEQ ID NO: 123 - DNA, 36 bp, Artificial Sequence
  IgG4 hinge region
gaaagcaaatacgggccgccgtgtccaccctgtccg